(19) United States
(12) Patent Application Publication
(10) Pub. No.: US 2024/0139322 A1
(43) Pub. Date: May 2, 2024

(54) COMPOSITIONS AND METHODS TO REDUCE THERAPEUTIC T CELL TOXICITY

(71) Applicant: H. LEE MOFFITT CANCER CENTER AND RESEARCH INSTITUTE INC., Tampa, FL (US)

(72) Inventors: Marco L. Davila, Tampa, FL (US); Gongbo Li, Chicago, IL (US)

(21) Appl. No.: 18/280,836

(22) PCT Filed: Mar. 10, 2022
(86) PCT No.: PCT/US2022/071070

§ 371 Date: Sep. 7, 2023

Related U.S. Application Data
(60) Provisional application No. 63/159,222, filed on Mar. 10, 2021.
(60) Provisional application No. 63/209,094, filed on Jun. 10, 2021.
(60) Provisional application No. 63/225,715, filed on Jul. 26, 2021.

(51) Int. Cl. A61K 39/00; A61P 35/00; C12N 5/0783; C12N 15/86

(52) U.S. Cl. CPC A61K 39/4633; A61K 39/4611; A61K 39/4613; A61K 39/4631; A61K 39/464404; A61K 39/464411; A61K 39/464429; A61K 39/464454; A61K 39/464466; A61K 39/464468; A61K 39/46447; A61K 39/464471; A61K 39/464482; A61K 39/464494; A61K 39/464495; A61P 35/00; C12N 5/0636; C12N 15/86; C12N 2501/51; C12N 2501/515; C12N 2740/10043

(57) ABSTRACT

Disclosed are off-the-shelf immune effector cells that are engineered to express anti-CD3 antibodies disclosed herein that are configured to autoactivate the immune effector cells, thereby decreasing expression of T cell receptors (e.g. TCRαβ) that could result in GVHD. Also disclosed are methods for modifying donor immune effector cells to make them suitable for off-the-shelf treatment of allogeneic subjects. These methods involve engineering the cells to express an anti-CD3 antibody configured to activate the cells. In some embodiments, the antibody is a bi-specific antibody that binds the CD3 complex on the immune effector cells. In other embodiments, the antibody is a membrane bound anti-CD3 antibody that autoactivates the immune effector cell.

### CROSS-REFERENCE TO RELATED APPLICATIONS

This application claims benefit of U.S. Provisional Application No. 63/159,222, filed Mar. 10, 2021, 63/209,094, filed Jun. 10, 2021, and 63/225,715, filed Jul. 26, 2021, which are hereby incorporated herein by reference in their entireties.

### SEQUENCE LISTING

This application contains a sequence listing filed in electronic form as an ASCII.txt file entitled “320803_2800_Sequence_Listing_ST25” created on Mar. 8, 2022, having 226,162 bytes. The content of the sequence listing is incorporated herein in its entirety.

### BACKGROUND

Autologous T cells, such as chimeric antigen receptor (CAR) T cells, have changed the therapeutic landscape in hematological malignancies. Nevertheless, the use of allogeneic T cells from donors has many potential advantages over autologous approaches, such as the immediate availability of cryopreserved batches for patient treatment, possible standardization of the T cell product, time for multiple cell modifications, redosing or combination of T cells directed against different targets, and decreased cost using an industrialized process. However, allogeneic T cells may cause life-threatening graft-versus-host disease and may be rapidly eliminated by the host immune system. Therefore, needed in the art are methods for modifying allogeneic T cells with decreased potential for graft-versus-host disease.

### SUMMARY

Disclosed are off-the-shelf immune effector cells that are engineered to express anti-CD3 antibodies disclosed herein that are configured to autoactivate the immune effector cells, thereby decreasing expression of T cell receptors (e.g. TCRαβ) that could result in GVHD.

Also disclosed are methods for modifying donor immune effector cells to make them suitable for off-the-shelf treatment of allogeneic subjects. These methods involve engineering the cells to express an anti-CD3 antibody configured to activate the cells. In some embodiments, the immune effector cell is further engineered to express a chimeric antigen receptor (CAR). In other embodiments, the immune effector cell does not express a CAR but instead relies on the antibody for targeting.

In some embodiments, the antibody is a bi-specific antibody that crosslinks the CD3 complex on the immune effector cells to another cell. In other embodiments, the antibody is a membrane bound anti-CD3 antibody that autoactivates the immune effector cell. In other embodiments, the antibody is a mono-specific antibody, such as an scFv.

Therefore, also disclosed is a method for enhancing CAR-T cells for allogeneic cell transfer, that involves engineering the CAR-T cells to secrete a monospecific anti-CD3 antibody.

In some embodiments, the immune effector cells are obtained from an allogeneic donor. Therefore, also disclosed is a method for treating cancer in a subject that involves obtaining immune effector cells from an allogeneic donor; engineering the immune effector cells to express an anti-CD3 multi-specific antibody, wherein the antibody is configured to bind a CD3 complex on the immune effector cells and a second antigen on another cell in a manner sufficient to activate the CD3 complex; and administering the engineered immune effector cell to the subject in an amount effective to treat the cancer.

In some embodiments, the immune effector cells comprise any CD3-expressing lymphocyte involved in defending the body against infectious disease and foreign materials.

Therefore, also disclosed are bispecific molecules that are able to crosslink CD3 complex on off-the-shelf immune effector cells with an antigen on another cell. The bispecific molecules can be engineered from fusion polypeptides comprising 1) variable domains of antibodies that specifically bind a target cell surface receptor and 2) variable domains of antibodies that specifically bind CD3. Either or both components of the bispecific molecules can also be engineered from non-antibody scaffolds including but not limited to nanobodies, monobodies, cyclic peptides, small molecules, and designed ankyrin repeat proteins (Darpins).

In some embodiments, the bispecific molecule is a Bispecific T-Cell Engaging (BiTE) antibody (fusion polypeptide) having, for example, the following formula:

SP-V_{L}R-V_{H}R-/-V_{L}3-V_{H}3,

SP-V_{H}R-V_{L}R-/-V_{H}3-V_{L}3,

SP-V_{L}R-V_{H}R-/-V_{H}3-V_{L}3,

SP-V_{H}R-V_{L}R-/-V_{L}3-V_{H}3,

SP-V_{L}3-V_{H}3-/-V_{L}R-V_{H}R,

SP-V_{H}3-V_{L}3-/-V_{H}R-V_{L}R,

SP-V_{L}3-V_{H}3-/-V_{H}R-V_{L}R, or

SP-V_{H}3-V_{L}3-/-V_{L}R-V_{H}R,
wherein “SP” represents an optional signal peptide,
wherein “V_{L}R” is a light chain variable domain specific for a target cell surface receptor;
wherein “V_{H}3” is a heavy chain variable domain specific for CD3;
wherein “V_{L}3” is a light chain variable domain specific for the CD3;
wherein “V_{H}R” is a heavy chain variable domain specific for the target cell surface receptor;
wherein “-” consists of a peptide linker or a peptide bond; and
wherein “-/-” consists of a peptide hinge sequence.

In some embodiments, the antibody is a bispecific antibody containing the full heavy and light chain regions. In this embodiment, the antibody may be generated by described methods such as the “knobs and holes” format (published in Ridgway JB, et al, Protein Eng. 1996 9(7):617-21).

The target cell surface receptor can in some cases be any other cell surface receptor, channel, or transporter expressed by a cell. In some embodiments, the receptor is a tumor associated antigen (TAA). Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, CSPG4, FAP, B7H3, Kit, CA LX, CS-1, MUC1, BCMA, bcr-abl, HER2, β-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-Ia, LMP2, NCAM, p53, p53 mutant, Ras mutant, gpIOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1 , ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, CD33, BCMA, GD2, CLL-1, CA-IX, MUCI, HER2, and any combination thereof. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

Also disclosed is an isolated nucleic acid encoding the disclosed fusion polypeptide, as well as nucleic acid vectors containing this isolated nucleic acid operably linked to an expression control sequence. Also disclosed are cells transfected with these vectors and the use of these cells to produce the disclosed fusion polypeptides.

A bi-specific antigen binding molecule can be formed from dimerization of heavy and light chains. In these embodiments, the V_{L}R dimerizes with V_{H}R to form an antigen binding site for a target cell surface receptor and the V_{H}3 dimerizes with V_{L}3 to form an antigen binding site for CD3.

Also disclosed is a bispecific antibody that is a single polypeptide chain comprising a bispecific antibody having a first antigen-binding region and a second antigen-binding region. In some cases, the first antigen-binding region is capable of specifically binding to the target receptor on the cell; and the second antigen-binding region is capable of specifically binding to CD3 on the immune effector cell.

Each of the first and second portions can comprise 1, 2, 3, or more antibody variable domains. In particular embodiments, each of the first and second portions contains two variable domains, a variable heavy (V_{H}) domain and a variable light (V_{L}) domain.

In some cases, the bispecific antibody has an affinity for the target receptor and CD3 corresponding to a K_{D} of about 10⁻⁷ M, 10⁻⁸ M, 10⁻⁹ M, or less.

Each of the first and second portions can be derived from natural antibodies, such as monoclonal antibodies. In some cases, the antibody is human. In some cases, the bispecific antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed is a pharmaceutical composition comprising a molecule disclosed herein in a pharmaceutically acceptable carrier. Also disclosed is a method for targeted ubiquitination of target receptors in a subject that involves administering to the subject a therapeutically effective amount of a disclosed pharmaceutical composition. Also disclosed is a kit comprising a bispecific antibody disclosed herein.

Also disclosed is an expression vector comprising an isolated nucleic acid encoding a bispecific antibody disclosed herein operably linked to an expression control sequence. Also disclosed is a cell comprising the disclosed expression vector. The cell can be a primary cell, transformed cell, cell line, or the like. In some cases, the cell is a mammalian cell line. In some cases, the cell is a non-mammalian cell line. For example, the cell can be a bacteria or insect cell line.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show CD3/TCRαβ expression in vitro after treatment with a CD19 BiTE or Her2 BiTE (FIG. 1A); Her2 BITE+αHer2BB, Her2 CAR+αCD3ϵIL15RA, or Her2 CAR+αCD3ϵ scFV (FIG. 1B); and CD19 BiTE+41BBLCD80, CD19 TriKE1 (aCD28), or CD19 TriKE2 (41BBL) (FIG. 1C).

FIGS. 2A and 2B show GvDH model test. FIG. 2A shows NSG mice treated with whole body irradiation and 1e7 Her2 BiTE-T or CD19 BiTE-T cells. FIG. 2B shows NSG mice treated with chemotherapy and 1e7 CTX+CAR-T or CTX+BiTE-T cells.

FIG. 3 shows CD3/TCRαβ expression in vivo without antigen. NSG mice received irradiation and then T cells. Donor T cells in peripheral blood were evaluated at certain time points. D12, Day 12 after T cell injection.

FIG. 4 shows CD3/TCRαβ expression in vivo with antigen. NSG mice were injected with tumor cells and then received T cell treatment. Donor T cells in peripheral blood or tumor were evaluated at certain time points.

FIGS. 5A to 5L show BiTE-T cells produce less cytokines and are comparable or better than CAR-T on efficacy in the presence of host T cells. Cytotoxicity (FIG. 5A) and cytokine production (FIG. 5B) of CD19 BiTE-T or CAR-T cells against 3T3.hCD19 cells. Cytotoxicity (FIG. 5C) and cytokine production (FIG. 5D) of Her2 BiTE-T or CAR-T cells against A375.Her2 cells. FIGS. 5E to 5H show a comparison study of CD19 BiTE-T and CAR-T cells in vivo with host T cell presence. FIG. 5E shows a study design. Male NSG mice received 1.2 Gy full body irradiation and then were engrafted with 7 million HLA-A2+ activated T cells one day after. Nalm6GL cells were given at day 0. Mice were treated with HLA-A2- CD19 BiTE-T or CAR-T cells at day 3. Survival (FIG. 5F), Donor T cells (FIG. 5G), and recipient T cells (FIG. 6H) in peripheral blood were monitored over time. FIGS. 5I to 5L show a comparison study of Her2 BiTE-T and CAR-T cells in vivo with host T cell presence. FIG. 5I shows a study design. Male NSG mice received 1.2 Gy full body irradiation and then were engrafted with 5 million HLA-A2+activated T cells one day after. A375.Her2 cells were given at day 0. Mice were treated with HLA-A2-Her2 BiTE-T or CAR-T cells at day 8. Tumor growth (FIG. 5J), Donor T cells (FIG. 5K), and recipient T cells (FIG. 5L) in peripheral blood were monitored over time. DTC, donor T cells; RTC, recipient T cells.

FIGS. 6A to 6O show co-stimulation or cytokines enhanced BiTE-T cells efficacy to match or overperform CAR-T cells. FIGS. 6A to 6C show a serial killing assay on A375.Her2 cells. FIG. 6A shows T cell proliferation over time. FIG. 6B shows TCRαβ expression over time. FIG. 6C shows CD3 expression over time. FIGS. 6D to 6H show efficacy comparison of co-stimulation enhanced Her2 BiTE-T and CAR-T cells. FIG. 6D show a study design. FIG. 6E show tumor growth. FIG. 6F show percentages of tumor infiltrating T cells. FIG. 6G show donor T cells in peripheral blood over time. FIG. 7H show weight change over time. FIGS. 6I to 6M show data from a comparison study of co-stimulation enhanced CD19 BiTE-T and CAR-T cells. FIG. 6I shows a study design. FIG. 6J shows survival. Small arrows indicate death events unrelated to leukemia or GvHD. FIG. 6K shows weight change. FIG. 6L shows donor T persistence in peripheral blood. FIG. 6M shows CD3/TCRαβ expression on WK 4 blood T cells. FIG. 6N (Her2) and FIG. 6O (CD19) show the evaluation of BiTE-T cells overexpressing IL7 and IL15 in serial killing assay in vitro.

FIGS. 7A to 7I show CD3 engagement induced TCRαβ/CD3 downregulation and BiTE engineered T cells have decreased alto-reactivity. FIG. 7A shows mouse TCR/CD3 expression on mouse T cells after treated with plate-coated mCD3ϵ antibody for 24 hr. FIG. 7B shows schematic diagrams of BiTE and CAR constructs used in the study. FIG. 7C shows TCRαβ evaluation on UT, BiTE-T or CAR-T cells using two different clones of TCRαβ antibodies. FIG. 7D shows TCRαβ/CD3 expression on CAR-T cells engineered with a membrane-bound or soluble secreted CD3ϵ scFv. FIG. 7E shows cytotoxicity of CAR-T cells engineered with a membrane-bound or soluble secreted CD3ϵ scFv. FIG. 7F shows immune phenotype of BiTE-T and CAR-T cells. FIG. 7G to 7I show a GvHD risk study using chemotherapy. Mice were treated with cyclophosphamide (CTX) at 250 mg/kg and then given 10 million of transduced T or untransduced T cells (UT). FIG. 7G shows survival. FIG. 7H shows weight change. FIG. 7I shows longterm leukemia protection by BiTE-T cells. In the same study, survived mice were challenged with NALM6GL cells 4.5 months after BiTE-T cells. Leukemia cells in peripheral blood were evaluated 3 weeks after challenge.

FIG. 8A shows representative flow plots of CD19 BiTE-T cells inducing CD3/TCRαβ downregulation on HLA-mismatched donor T cells. FIG. 8B shows Her2 BiTE-T cells reduced CD3/TCRαβ expression on allogeneic T cells. HLA-A2+ Her2 BiTE-T cells were co-cultured with HLA-A2-PBMCs for 5 days without cytokines. Total cells were subjected to flow analysis.

FIGS. 9A to 9H show BiTE-T cells are not as durable as CAR-T cells. FIG. 10A shows T cell expansion in CD19 serial killing assay. FIG. 9B shows NALM6GL cell killing in serial killing assay. FIG. 9C shows T cell expansion in A375.Her2 serial killing assay. FIG. 9D shows A375.Her2 cell killing in serial killing assay. FIG. 10E (survival) & FIG. 9F (T cell persistence) show in vivo efficacy comparison of CD19 BiTE-T and CAR-T cells. FIG. 9G (tumor growth) & FIG. 9H (T cell persistence) show in vivo efficacy comparison of Her2 BiTE-T and CAR-T cells.

FIG. 10 shows bioluminescence data of co-stimulation enhanced CD19 BiTE-T cells compared to CD19 CAR-T cells.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “bispecific antibody” refers to an antibody having two different antigen-binding regions defined by different antibody sequences. This can be understood as different target binding but includes as well binding to different epitopes in one target.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

The term “isolated nucleic acid” refers to a polynucleotide of genomic, cDNA, synthetic, or natural origin or some combination thereof, which (1) is not associated with the cell in which the “isolated nucleic acid” is found in nature, or (2) is operably linked to a polynucleotide to which it is not linked in nature.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ϵ-amino caproic acid, 7-amino heptanoic acid, L-aspartic acid, L-glutamic acid, N-ϵ-Boc-N-α-CBZ-L-lysine, N-ϵ-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The term “protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” is meant a molecule that is produced as a result of transcription of a gene. Gene products include RNA molecules transcribed from a gene, as well as proteins translated from such transcripts.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 1^{0 10} M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Off-the-Shelf Immune Effector Cells

Disclosed are off-the-shelf immune effector cells that are engineered to express anti-CD3 antibodies disclosed herein that are configured to autoactivate the immune effector cells, thereby decreasing expression of T cell receptors (e.g. TCRαβ) that could result in GVHD. These cells are therefore preferably obtained from a healthy donor subject. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes, preferably cytotoxic T lymphocytes (CTLs).

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan RA, et al. Mol Ther 2010 18:843-851), tumor lysis syndrome (Porter DL, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Epstein-Barr virus (EBV)-induced lymphoproliferative diseases (EBV-LPDs) and other EBV-associated cancers are a significant cause of morbidity and mortality for recipients of allogeneic hematopoietic cell transplantation (HCT) or solid organ transplants (SOT), particularly in those who have received certain T-cell reactive Abs to prevent or treat GVHD. Prophylaxis and treatment by the adoptive transfer of autologous or allogeneic EBV-specific cytotoxic T cells and the subsequent long-term restoration of immunity against EBV-associated lymphoproliferation have provided positive outcomes in the management of these uniformly fatal complications of allogeneic tissue transfer. Therefore, in some embodiments, the disclosed immune effector cells that comprise one or more of the CAR polypeptides of the present invention are allogeneic or autologous EBV-specific cytotoxic T lymphocytes (CTLs). For example, generation of EBV-specific cytotoxic T cells may involve isolating PBMCs from of an EBV-seropositive autologous or allogenic donor and enriching them for T cells by depletion of monocytes and NK cells. EBV-specific cytotoxic T cells may also be produced by contacting donor PBMCs or purified donor T cells with a “stimulator” cell that expresses one or more EBV antigen(s) and presents the EBV antigen(s) to unstimulated T cells, thereby causing stimulation and expansion of EBV-specific CTLs. EBV antigens include, for example, latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. Cytotoxic T cells that comprise T cell receptor(s) which recognize one or more EBV-specific antigens are deemed to have been “sensitized” to those EBV antigen(s) and are therefore termed “EBV-sensitized cytotoxic T cells” herein. Known methods for generating allogeneic or autologous EBV-specific cytotoxic T cell populations that may comprise one or more of the CAR polypeptides of the present invention are described, for example, in Barker et al., Blood 2010 116(23):5045-49; Doubrovina, et al., Blood 2012 119(11):2644-56; Koehne, et al. Blood 2002 99(5):1730-40; and Smith et al. Cancer Res 2012 72(5):1116-25, which are incorporated by reference for these teachings.

### Bispecific Antibodies

Bispecific antibodies may contain a heavy chain comprising one or more variable regions and/or a light chain comprising one or more variable regions. Bispecific antibodies can be constructed using only antibody variable domains. A fairly efficient and relatively simple method is to make the linker sequence between the V_{H} and V_{L} domains so short that they cannot fold over and bind one another. Reduction of the linker length to 3-12 residues prevents the monomeric configuration of the scFv molecule and favors intermolecular VH-VL pairings with formation of a 60 kDa non-covalent scFv dimer “diabody”. The diabody format can also be used for generation of recombinant bi-specific antibodies, which are obtained by the noncovalent association of two single-chain fusion products, consisting of the VH domain from one antibody connected by a short linker to the VL domain of another antibody. Reducing the linker length still further below three residues can result in the formation of trimers (“triabody”, about 90 kDa) or tetramers (“tetrabody”, about 120 kDa). For a review of engineered antibodies, particularly single domain fragments, see Holliger and Hudson, 2005, Nature Biotechnology, 23:1126-1136. All of such engineered antibodies may be used in the fusion polypeptides provided herein.

Peptide linkers (-) suitable for production of scFv antibodies are described in Kumada Y, et al. Biochemical Engineering Journal. 2007 35(2):158-165; Albrecht H, et al. J Immunol Methods. 2006 310(1-2):100-16; Feng J, et al. J Immunol Methods. 2003 282(1-2):33-43; Griffiths AD, et al. Curr Opin Biotechnol. 1998 9(1):102-8; Huston JS, et al. Methods Enzymol. 1991 203:46-88; Bird RE, et al. Science. 1988 242(4877):423-6; Takkinen K, et al. Protein Eng. 1991 4(7):837-41; Smallshaw JE, et al. Protein Eng. 1999 12(7):623-30; Argos P. J Mol Biol. 1990 211(4):943-58; and Whitlow M, et al. Protein Eng. 1993 6(8):989-95, which are hereby incorporated by reference for the teachings of these linkers and methods of producing scFv antibodies against different targets using various linkers.

Tetravalent Tandab® may be prepared substantially as described in WO 1999/057150 A3 or US2006/0233787, which are incorporated by reference for the teaching of methods of making Tandab® molecules.

The antigen recognition sites or entire variable regions of the engineered antibodies may be derived from one or more parental antibodies directed against any antigen of interest (e.g., target receptor ECD or TMUL ECD). The parental antibodies can include naturally occurring antibodies or antibody fragments, antibodies or antibody fragments adapted from naturally occurring antibodies, antibodies constructed de novo using sequences of antibodies or antibody fragments known to be specific for an antigen of interest. Sequences that may be derived from parental antibodies include heavy and/or light chain variable regions and/or CDRs, framework regions or other portions thereof.

In some cases, the V_{L}3 comprises the amino acid sequence MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRA (SEQ ID NO: 1), or a fragment or variant thereof able to bind CD3 having at least 70%, 75%, 80%, 85%, 90%, 95%, or 100% sequence identity to SEQ ID NO: 1. In some cases, the V_{H}3 comprises the amino acid sequence EVQLQQSGPELVKPGASM KISCKASGYSFTGYTMNWVKQSHGKNLEWMGLINPYK GVSTYNQKFKDKATLTVDKSSSTAYMELLSLTSEDSAVYYCARSGYYGDSDWYFD VWGAGTTVTV (SEQ ID NO: 2), or a fragment or variant thereof able to bind CD3 having at least 70%, 75%, 80%, 85%, 90%, 95%, or 100% sequence identity to SEQ ID NO: 2.

Other anti-CD3 antibody sequences are known in the art, such as OKT3, which can be used in the disclosed system.

In some cases, the target cell surface receptor is Her2. Therefore, in some cases, the V_{H}R comprises the amino acid sequence EVQLVESGGGLVQPGGSLRLSCAASGFNIKDTYIHWVRQAPGKGLEWVARIYPTN GYTRYADSVKGRFTISADTSKNTAYLQMNSLRAEDTAVYYCSRWGGDGFYAMDV WGQGTLVTVSSEPKSCDKTHTCP (SEQ ID NO: 3), or a fragment or variant thereof able to bind Her2 having at least 70%, 75%, 80%, 85%, 90%, 95%, or 100% sequence identity to SEQ ID NO: 3. In some cases, the V A R comprises the amino acid sequence DIQMTQSPSSLSASVGDRVTITCRASQDVNTAVAWYQQKPGKAPKWYSASFLYS GVPSRFSGSRSGTDFTLTISSLQPEDFATYYCQQHYTTPPTFGQGTKVEIK (SEQ ID NO: 4), or a fragment or variant thereof able to bind Her2 having at least 70%, 75%, 80%, 85%, 90%, 95%, or 100% sequence identity to SEQ ID NO: 4.

In some cases, the target cell surface receptor is EGFR. In some cases, the VHR comprises the amino acid sequence QIQLVQSGPELKKPGETVKISCKASGYTFTEYPIHWVKQAPGKGFKWMGMIYTDIG KPTYAEEFGRFAFSLETSASTAYLQINNLKNEDTATYFCVRDRYDSLFDYWGQGTT LTVSS (SEQ ID NO: 5), or a fragment or variant thereof able to bind EGFR having at least 70%, 75%, 80%, 85%, 90%, 95%, or 100% sequence identity to SEQ ID NO: 5. In some cases, the V_{L}R comprises the amino acid sequence DVVMTQTPLSLPVSLGDQASISCRSSQSLVHSNGNTYLHWYLQKPGQSPKWYKV SNRFSGVPDRFSGSGSGTDFTLKISRVEAEDLGVYFCSQSTHVPWTFGGGTKLEIK (SEQ ID NO: 6), or a fragment or variant thereof able to bind EGFR having at least 70%, 75%, 80%, 85%, 90%, 95%, or 100% sequence identity to SEQ ID NO: 6.

In some cases, the target cell surface receptor is CSPG4. In some cases, the V_{H}R comprises the amino acid sequence EVQLVESGAEVKKPGDSLKISCKGSGYSFTSYWIGWVRQMPGKGLEWMGIIYPGD SETTYSPAFQGDVTISVDKSISTAYLQWNSLKASDTGIYYCARRRGNYYMDVWGNG TLVTVSSLKS (SEQ ID NO: 7), or a fragment or variant thereof able to bind CSPG4 having at least 70%, 75%, 80%, 85%, 90%, 95%, or 100% sequence identity to SEQ ID NO: 7. In some cases, the V_{L}R comprises the amino acid sequence RSTQSALTQPASVSGSPGQSITISCTGTSSDVGGYNYVSWYQQHPGKAPKLMIYDV SNRPSGVSNRFSGSKSGNTASLTISGLQAEDEADYYCSSYTSSSTRHVFGTGTQLT VLG (SEQ ID NO:8), or a fragment or variant thereof able to bind CSPG4 having at least 70%, 75%, 80%, 85%, 90%, 95%, or 100% sequence identity to SEQ ID NO: 8.

In some cases, the target cell surface receptor is CD20. In some cases, the V_{H}R comprises the amino acid sequence QVQLQQPGAELVKPGASVKMSCKASGYTFTSYNMHWVKQTPGRGLEWIGAIYPG NGDTSYNQKFKGKATLTADKSSSTAYMQLSSLTSEDSAVYYCARSTYYGGDWYFN VWGAGTTVTVSA (SEQ ID NO:9), or a fragment or variant thereof able to bind CSPG4 having at least 70%, 75%, 80%, 85%, 90%, 95%, or 100% sequence identity to SEQ ID NO:9. In some cases, the V_{L}R comprises the amino acid sequence QIVLSQSPAILSASPGEKVTMTCRASSSVSYIHWFQQKPGSSPKPWIYATSNLASGV PVRFSGSGSGTSYSLTISRVEAEDAATYYCQQWTSNPPTFGGGTKLEIK (SEQ ID NO:10), or a fragment or variant thereof able to bind CSPG4 having at least 70%, 75%, 80%, 85%, 90%, 95%, or 100% sequence identity to SEQ ID NO:10.

In some cases, the target cell surface receptor is CD20. In some cases, the V_{H}R comprises the amino acid sequence EVQLQQSGAELVKPGASVKMSCKASGYTFTSYNMHWVKQTPGQGLEWIGAIYPG NGDTSYNQKFKG KATLTADKSSSTAYMQLSSLTSEDSADYYCARSNYYGSSYWFF DVWGAGTTVTVSS (SEQ ID NO:11), or a fragment or variant thereof able to bind CSPG4 having at least 70%, 75%, 80%, 85%, 90%, 95%, or 100% sequence identity to SEQ ID NO:11. In some cases, the V_{L}R comprises the amino acid sequence DIVLTQSPAILSASPGEKVTMTCRASSSVNYMDWYQKKPGSSPKPWIYATSNLASG VPARFSGSGSGTSYSLTISRVEAEDAATYYCQQWSFNPPTFGGGTKLEIK (SEQ ID NO:12), or a fragment or variant thereof able to bind CSPG4 having at least 70%, 75%, 80%, 85%, 90%, 95%, or 100% sequence identity to SEQ ID NO:12.

In some cases, the target cell surface receptor is PSMA. In some cases, the V_{H}R comprises the amino acid sequence MARFSSSSLDLNWYSLGLQXKLSCKASGYTFTYFDINWLRQRPEQGLEWIGVISPG DGNTNYNENFKGKATLTIDKSSTTAYIQLSRLTSEDSAVYFCARDGNFPYYAMDSW GQGTSVTVSSAKTTPKL (SEQ ID NO:13), or a fragment or variant thereof able to bind CSPG4 having at least 70%, 75%, 80%, 85%, 90%, 95%, or 100% sequence identity to SEQ ID NO:13. In some cases, the V_{L}R comprises the amino acid sequence DIVMTQIPLSLPVILGDQASISCRSSQSLVYSNGNTYLHWFLQKPGQSPKLLIYNVSN LFSGVPDRFSGSGSGTDFTLKISRVEAEDLGIYFCSQSTHVPTFGGGTKLEIKRADA AAAGS (SEQ ID NO:14), or a fragment or variant thereof able to bind CSPG4 having at least 70%, 75%, 80%, 85%, 90%, 95%, or 100% sequence identity to SEQ ID NO:14.

In some cases, the target cell surface receptor is BCMA. In some cases, the V_{H}R comprises the amino acid sequence QLQLQESGPGLVKPSETLSLTCTVSGGSISSGSYFWGWIRQPPGKGLEWIGSIYYS GITYYNPSLKSRVTISVDTSKNQFSLKLSSVTAADTAVYYCARHDGAVAGLFDYWG QGTLVTVSSA (SEQ ID NO:15), or a fragment or variant thereof able to bind CSPG4 having at least 70%, 75%, 80%, 85%, 90%, 95%, or 100% sequence identity to SEQ ID NO:15. In some cases, the V_{L}R comprises the amino acid sequence YVLTQPPSVSVAPGQTARITCGGNNIGSKSVHWYQQPPGQAPVVVVYDDSDRPSG IPERFSGSNSGNTATLTISRVEAGDEAVYYCQVWDSSSDHVVFGGGTKLTVL (SEQ ID NO:16), or a fragment or variant thereof able to bind CSPG4 having at least 70%, 75%, 80%, 85%, 90%, 95%, or 100% sequence identity to SEQ ID NO:16.

In some cases, the target cell surface receptor is Mesothelin. In some cases, the V_{H}R comprises the amino acid sequence QVQLVQSGAEVKRPGASVQVSCRASGYSINTYYMQWVRQAPGAGLEWMGVINPS GVTSYAQKFQGRVTLTNDTSTNTVYMQLNSLTSADTAVYYCARWALWGDFGMDV WGKGTLVTVSS (SEQ ID NO:17), or a fragment or variant thereof able to bind CSPG4 having at least 70%, 75%, 80%, 85%, 90%, 95%, or 100% sequence identity to SEQ ID NO:17. In some cases, the V_{L}R comprises the amino acid sequence DIQMTQSPSTLSASIGDRVTITCRASEGIYHWLAWYQQKPGKAPKLLIYKASSLASG APSRFSGSGSGTDFTLTISSLQPDDFATYYCQQYSNYPLTFGGGTKLEIK (SEQ ID NO:18), or a fragment or variant thereof able to bind CSPG4 having at least 70%, 75%, 80%, 85%, 90%, 95%, or 100% sequence identity to SEQ ID NO:18.

In some cases, the target cell surface receptor is GPC3. In some cases, the V_{H}R comprises the amino acid sequence QVQLVQSGGGVVQPGRSLRLSCAASGFTFSSYGLHWVRQAPGKGLEWYAAISYD GSKKYYADSVKGRLTISRDNSKNTLYLQM NSLSPEDTALYFCARGWFVEPLSWGQ GTLVTVSS (SEQ ID NO:19), or a fragment or variant thereof able to bind CSPG4 having at least 70%, 75%, 80%, 85%, 90%, 95%, or 100% sequence identity to SEQ ID NO:19. In some cases, the V_{L}R comprises the amino acid sequence QSVLTQPPSASGTPGQRVTISCSGSSSNIGSNTVNWYQQLPGTAPKLLIYSNNQRP SGVPDRFSGSKSGTSASLAISGLQSEDEADYYCAAWDDSLNGYVFGTGTKLTVL (SEQ ID NO:20), or a fragment or variant thereof able to bind CSPG4 having at least 70%, 75%, 80%, 85%, 90%, 95%, or 100% sequence identity to SEQ ID NO:20.

In some cases, the target cell surface receptor is EpCAM. In some cases, the V_{H}R comprises the amino acid sequence QVQLVQSGAEVKKPGSSVRVSCKASGGTFSSYAISWVRQAPGQGLEWMGGIIPIF GTANYAQKFQGRVTITADESTSTAYMELSSLRSEDTAVYYCARDPFLHYWGQGTLV T (SEQ ID NO:21), or a fragment or variant thereof able to bind CSPG4 having at least 70%, 75%, 80%, 85%, 90%, 95%, or 100% sequence identity to SEQ ID NO:21. In some cases, the V_{L}R comprises the amino acid sequence EIELTQSPLSLPVTPGEPASISCRSSQSLLHSNGYNYLDWYLQKPGQSPQLLIYLGS NRASGVPDRFSGSGSGTDFTLKISRVEAEDVGVYYCMQALQTFTFGPGTKVEI (SEQ ID NO:22), or a fragment or variant thereof able to bind CSPG4 having at least 70%, 75%, 80%, 85%, 90%, 95%, or 100% sequence identity to SEQ ID NO:22.

In some cases, the target cell surface receptor is GD2. In some cases, the V_{H}R comprises the amino acid sequence QVQLVESGPGVVQPGRSLRISCAVSGFSVTNYGVHWVRQPPGKGLEWLGVIWAG GITNYNSAFMSRLTISKDNSKNTVYLQMNSLRAEDTAMYYCASRGGHYGYALDYW GQGTLVTVSS (SEQ ID NO:23), or a fragment or variant thereof able to bind CSPG4 having at least 70%, 75%, 80%, 85%, 90%, 95%, or 100% sequence identity to SEQ ID NO:23. In some cases, the V A R comprises the amino acid sequence EIVMTQTPATLSVSAGERVTITCKASQSVSNDVTWYQQKPGQAPRLLIYSASNRYS GVPARFSGSGYGTEFTFTISSVQSEDFAVYFCQQDYSSFGQGTKLEIKR (SEQ ID NO:24), or a fragment or variant thereof able to bind CSPG4 having at least 70%, 75%, 80%, 85%, 90%, 95%, or 100% sequence identity to SEQ ID NO:24.

The particular length of the peptide linker (--) used to join the scFv molecules together is important in determining half-life, immunogenicity, and activity of the overall construct. In some embodiments, the linker sequence (--) is 4, 5, 6, 7, 8, 9, 10, 11, 12, 13, 14, 15, 16, 17, 18, 19, 20, 21, 22, 23, 24, 25 or more amino acids in length. In some embodiments, the linker sequence (--) comprises GGGGS (SEQ ID NO:25). In some cases, the linker comprises 2, 3, 4, 5, or more GGGGS (SEQ ID NO:25) sequences.

In some embodiments, the hinge sequence (-/-) comprises EPKSCDKTHTCP (SEQ ID NO:26). The linker is preferably long enough to not interfere with proper folding and association of the V_{H}-V_{L} chains but not so long as to cause added immunogenicity.

Therefore, in some embodiments, the bi-specific antibody has the amino acid sequence:
DIQMTQSPSSLSASVGDRVTITCRASQDVNTAVAWYQQKPGKAPKWYSASFLYS GVPSRFSGSRSGTDFTLTISSLQPEDFATYYCQQHYTTPPTFGQGTKVEIKRTGSTS GSGKPGSGEGSEVQLVESGGGLVQPGGSLRLSCAASGFNIKDTYIHWVRQAPGKG LEWVARIYPTNGYTRYADSVKGRFTISADTSKNTAYLQMNSLRAEDTAVYYCSRWG GDGFYAM DVWGQGTLVTVSSEPKSCDKTHTCPEPKSCDKTHTCPMADIQMTQTT SSLSASLGDRVTISCRASQDIRNYLNWYQQKPDGTVKLLIYYTSRLHSGVPSKFSGS GSGTDYSLTISNLEQEDIATYFCQQGNTLPWTFAGGTKLEIKRAGGGSGGGSGGG SGGGSEVQLQQSGPELVKPGASM KISCKASGYSFTGYTMNWVKQSHGKNLEWM GLINPYKGVSTYNQKFKDKATLTVDKSSSTAYM ELLSLTSEDSAVYYCARSGYYGD SDWYFDVWGAGTTVTV (SEQ ID NO:27), or a fragment or variant thereof able to bind CD3 and Her2 having at least 70%, 75%, 80%, 85%, 90%, 95%, or 100% sequence identity to SEQ ID NO:27.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASM KISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPDIQMTQSPSSLSASVGD RVTITCRASQDVNTAVAWYQQKPGKAPKWYSASFLYSGVPSRFSGSRSGTDFTL TISSLQPEDFATYYCQQHYTTPPTFGQGTKVEIKRTGSTSGSGKPGSGEGSEVQLV ESGGGLVQPGGSLRLSCAASGFNIKDTYIHWVRQAPGKGLEWVARIYPTNGYTRY ADSVKGRFTISADTSKNTAYLQMNSLRAEDTAVYYCSRWGGDGFYAMDVWGQGT LVTVSS (SEQ ID NO:28), or a fragment or variant thereof able to bind CD3 and Her2 having at least 70%, 75%, 80%, 85%, 90%, 95%, or 100% sequence identity to SEQ ID NO:28.

Therefore, in some embodiments, the bi-specific antibody has the amino acid sequence:
QIQLVQSGPELKKPGETVKISCKASGYTFTEYPIHWVKQAPGKGFKWMGMIYTDIG KPTYAEEFGRFAFSLETSASTAYLQINNLKNEDTATYFCVRDRYDSLFDYWGQGTT LTVSSGGGGSGGGGSGGGGSDVVMTQTPLSLPVSLGDQASISCRSSQSLVHSNG NTYLHWYLQKPGQSPKWYKVSNRFSGVPDRFSGSGSGTDFTLKISRVEAEDLGV YFCSQSTHVPWTFGGGTKLEIKEPKSCDKTHTCPEPKSCDKTHTCPMADIQMTQTT SSLSASLGDRVTISCRASQDIRNYLNWYQQKPDGTVKLLIYYTSRLHSGVPSKFSGS GSGTDYSLTISNLEQEDIATYFCQQGNTLPWTFAGGTKLEIKRAGGGSGGGSGGG SGGGSEVQLQQSGPELVKPGASM KISCKASGYSFTGYTMNWVKQSHGKNLEWM GLINPYKGVSTYNQKFKDKATLTVDKSSSTAYMELLSLTSEDSAVYYCARSGYYGD SDWYFDVWGAGTTVTV (SEQ ID NO:29), or a fragment or variant thereof able to bind CD3 and EGFR having at least 70%, 75%, 80%, 85%, 90%, 95%, or 100% sequence identity to SEQ ID NO:29.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQIQLVQSGPELKKPGETV KISCKASGYTFTEYPIHWVKQAPGKGFKWMGMIYTDIGKPTYAEEFGRFAFSLETSA STAYLQINNLKNEDTATYFCVRDRYDSLFDYWGQGTTLTVSSGGGGSGGGGSGG GGSDVVMTQTPLSLPVSLGDQASISCRSSQSLVHSNGNTYLHWYLQKPGQSPKLLI YKVSNRFSGVPDRFSGSGSGTDFTLKISRVEAEDLGVYFCSQSTHVPWTFGGGTK LEIKEPKSCDKTHTCP (SEQ ID NO:30), or a fragment or variant thereof able to bind CD3 and EGFR having at least 70%, 75%, 80%, 85%, 90%, 95%, or 100% sequence identity to SEQ ID NO:30.

Therefore, in some embodiments, the bi-specific antibody has the amino acid sequence:
EVQLVESGAEVKKPGDSLKISCKGSGYSFTSYWIGWVRQMPGKGLEWMGIIYPGD SETTYSPAFQGDVTISVDKSISTAYLQWNSLKASDTGIYYCARRRGNYYMDVWGNG TLVTVSSLKSGGGGSGGGGSGGGGSRSTQSALTQPASVSGSPGQSITISCTGTSS DVGGYNYVSWYQQHPGKAPKLMIYDVSNRPSGVSNRFSGSKSGNTASLTISGLQA EDEADYYCSSYTSSSTRHVFGTGTQLTVLGEPKSCDKTHTCPEPKSCDKTHTCPM ADIQMTQTTSSLSASLGDRVTISCRASQDIRNYLNWYQQKPDGTVKLLIYYTSRLHS GVPSKFSGSGSGTDYSLTISNLEQEDIATYFCQQGNTLPWTFAGGTKLEIKRAGGG SGGGSGGGSGGGSEVQLQQSGPELVKPGASMKISCKASGYSFTGYTM NWVKQS HGKNLEWMGLINPYKGVSTYNQKFKDKATLTVDKSSSTAYMELLSLTSEDSAVYYC ARSGYYGDSDWYFDVWGAGTTVTV (SEQ ID NO:31), or a fragment or variant thereof able to bind CD3 and CSPG4 having at least 70%, 75%, 80%, 85%, 90%, 95%, or 100% sequence identity to SEQ ID NO:31.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPEVQLVESGAEVKKPGDS LKISCKGSGYSFTSYWIGWVRQMPGKGLEWMGIIYPGDSETTYSPAFQGDVTISVD KSISTAYLQWNSLKASDTGIYYCARRRGNYYMDVWGNGTLVTVSSLKSGGGGSGG GGSGGGGSRSTQSALTQPASVSGSPGQSITISCTGTSSDVGGYNYVSWYQQHPG KAPKLMIYDVSNRPSGVSNRFSGSKSGNTASLTIG LQAEDEADYYCSSYTSSSTR HVFGTGTQLTVLGEPKSCDKTHTCP (SEQ ID NO:32), or a fragment or variant thereof able to bind CD3 and CSPG4 having at least 70%, 75%, 80%, 85%, 90%, 95%, or 100% sequence identity to SEQ ID NO:32.

Therefore, in some embodiments, the bi-specific antibody has the amino acid sequence:
QVQLQQPGAELVKPGASVKMSCKASGYTFTSYNMHWVKQTPGRGLEWIGAIYPG NGDTSYNQKFKGKATLTADKSSSTAYMQLSSLTSEDSAVYYCARSTYYGGDWYFN VWGAGTTVTVSAGGGGSGGGGSGGGGSQIVLSQSPAILSASPGEKVTMTCRASS SVSYIHWFQQKPGSSPKPWIYATSNLASGVPVRFSGSGSGTSYSLTISRVEAEDAA TYYCQQWTSNPPTFGGGTKLEIKEPKSCDKTHTCPMADIQMTQTTSSLSASLGDRV TISCRASQDIRNYLNWYQQKPDGTVKLLIYYTSRLHSGVPSKFSGSGSGTDYSLTIS NLEQEDIATYFCQQGNTLPWTFAGGTKLEIKRAGGGSGGGSGGGSGGGSEVQLQ QSGPELVKPGASMKISCKASGYSFTGYTMNWVKQSHGKNLEWMGLINPYKGVSTY NQKFKDKATLTVDKSSSTAYMELLSLTSEDSAVYYCARSGYYGDSDWYFDVWGAG TTVTV (SEQ ID NO:33), or a fragment or variant thereof able to bind CD3 and CD20 having at least 70%, 75%, 80%, 85%, 90%, 95%, or 100% sequence identity to SEQ ID NO:33.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGI PYKGVSTYNQKFKDKATLTVDKSSSTAYM ELLSLTSEDSAVYY CARSGYYGDSDWYFDVWGAGTTVTVEPKSCDKTHTCPQVQLQQPGAELVKPGAS VKMSCKASGYTFTSYNMHWVKQTPGRGLEWIGAIYPGNGDTSYNQKFKGKATLTA DKSSSTAYMQLSSLTSEDSAVYYCARSTYYGGDWYFNVWGAGTTVTVSAGGGGS GGGGSGGGGSQIVLSQSPAILSASPGEKVTMTCRASSSVSYIHWFQQKPGSSPKP WIYATSNLASGVPVRFSGSGSGTSYSLTISRVEAEDAATYYCQQWTSNPPTFGGGT KLEIK (SEQ ID NO:34), or a fragment or variant thereof able to bind CD3 and CD20 having at least 70%, 75%, 80%, 85%, 90%, 95%, or 100% sequence identity to SEQ ID NO:34.

Therefore, in some embodiments, the bi-specific antibody has the amino acid sequence:
EVQLQQSGAELVKPGASVKMSCKASGYTFTSYNMHWVKQTPGQGLEWIGAIYPG NGDTSYNQKFKGKATLTADKSSSTAYMQLSSLTSEDSADYYCARSNYYGSSYWFF DVWGAGTTVTVSSGGGGSGGGGSGGGGSDIVLTQSPAILSASPGEKVTMTCRAS SSVNYMDWYQKKPGSSPKPWIYATSNLASGVPARFSGSGSGTSYSLTISRVEAED AATYYCQQWSFNPPTFGGGTKLEIKEPKSCDKTHTCPMADIQMTQTTSSLSASLGD RVTISCRASQDIRNYLNWYQQKPDGTVKLLIYYTSRLHSGVPSKFSGSGSGTDYSLT ISNLEQEDIATYFCQQGNTLPWTFAGGTKLEIKRAGGGSGGGSGGGSGGGSEVQL QQSGPELVKPGASMKISCKASGYSFTGYTMNWVKQSHGKNLEWMGLINPYKGVS TYNQKFKDKATLTVDKSSSTAYMELLSLTSEDSAVYYCARSGYYGDSDWYFDVWG AGTTVTV (SEQ ID NO:35), or a fragment or variant thereof able to bind CD3 and CD20 having at least 70%, 75%, 80%, 85%, 90%, 95%, or 100% sequence identity to SEQ ID NO:35.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPEVQLQQSGAELVKPGAS VKMSCKASGYTFTSYNMHWVKQTPGQGLEWIGAIYPGNGDTSYNQKFKGKATLTA DKSSSTAYMQLSSLTSEDSADYYCARSNYYGSSYWFFDVWGAGTTVTVSSGGGG SGGGGSGGGGSDIVLTQSPAILSASPGEKVTMTCRASSSVNYMDWYQKKPGSSPK PWIYATSNLASGVPARFSGSGSGTSYSLTISRVEAEDAATYYCQQWSFNPPTFGG GTKLEIK (SEQ ID NO:36), or a fragment or variant thereof able to bind CD3 and CD20 having at least 70%, 75%, 80%, 85%, 90%, 95%, or 100% sequence identity to SEQ ID NO:36.

Therefore, in some embodiments, the bi-specific antibody has the amino acid sequence:
MARFSSSSLDLNWYSLGLQXKLSCKASGYTFTYFDINWLRQRPEQGLEWIGVISPG DGNTNYNENFKGKATLTIDKSSTTAYIQLSRLTSEDSAVYFCARDGNFPYYAMDSW GQGTSVTVSSAKTTPKLEEGEFSEARVDIVMTQIPLSLPVILGDQASISCRSSQSLVY SNGNTYLHWFLQKPGQSPKLLIYNVSNLFSGVPDRFSGSGSGTDFTLKISRVEAED LGIYFCSQSTHVPTFGGGTKLEIKRADAAAAGSEPKSCDKTHTCPMADIQMTQTTS SLSASLGDRVTISCRASQDIRNYLNWYQQKPDGTVKLLIYYTSRLHSGVPSKFSGSG SGTDYSLTISNLEQEDIATYFCQQGNTLPWTFAGGTKLEIKRAGGGSGGGSGGGS GGGSEVQLQQSGPELVKPGASMKISCKASGYSFTGYTMNWVKQSHGKNLEWMGL INPYKGVSTYNQKFKDKATLTVDKSSSTAYMELLSLTSEDSAVYYCARSGYYGDSD WYFDVWGAGTTVTV (SEQ ID NO:37), or a fragment or variant thereof able to bind CD3 and PSMA having at least 70%, 75%, 80%, 85%, 90%, 95%, or 100% sequence identity to SEQ ID NO:37.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPMARFSSSSLDLNWYSLG LQXKLSCKASGYTFTYFDINWLRQRPEQGLEWIGVISPGDGNTNYNENFKGKATLTI DKSSTTAYIQLSRLTSEDSAVYFCARDGNFPYYAMDSWGQGTSVTVSSAKTTPKLE EGEFSEARVDIVMTQIPLSLPVILGDQASISCRSSQSLVYSNGNTYLHWFLQKPGQS PKLLIYNVSNLFSGVPDRFSGSGSGTDFTLKISRVEAEDLGIYFCSQSTHVPTFGGG TKLEIKRADAAAAGS (SEQ ID NO:38), or a fragment or variant thereof able to bind CD3 and PSMA having at least 70%, 75%, 80%, 85%, 90%, 95%, or 100% sequence identity to SEQ ID NO:38.

Therefore, in some embodiments, the bi-specific antibody has the amino acid sequence:
QLQLQESGPGLVKPSETLSLTCTVSGGSISSGSYFWGWIRQPPGKGLEWIGSIYYS GITYYNPSLKSRVTISVDTSKNQFSLKLSSVTAADTAVYYCARHDGAVAGLFDYWG QGTLVTVSSAGGGGSGGGGSGGGGSSYVLTQPPSVSVAPGQTARITCGGNNIGS KSVHWYQQPPGQAPVVVVYDDSDRPSGIPERFSGSNSGNTATLTISRVEAGDEAV YYCQVWDSSSDHVVFGGGTKLTVLEPKSCDKTHTCPMADIQMTQTTSSLSASLGD RVTISCRASQDIRNYLNWYQQKPDGTVKLLIYYTSRLHSGVPSKFSGSGSGTDYSLT ISNLEQEDIATYFCQQGNTLPWTFAGGTKLEIKRAGGGSGGGSGGGSGGGSEVQL QQSGPELVKPGASMKISCKASGYSFTGYTMNWVKQSHGKNLEWMGLINPYKGVS TYNQKFKDKATLTVDKSSSTAYMELLSLTSEDSAVYYCARSGYYGDSDWYFDVWG AGTTVTV (SEQ ID NO:39), or a fragment or variant thereof able to bind CD3 and BCMA having at least 70%, 75%, 80%, 85%, 90%, 95%, or 100% sequence identity to SEQ ID NO:39.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQLQLQESGPGLVKPSET LSLTCTVSGGSISSGSYFWGWIRQPPGKGLEWIGSIYYSGITYYNPSLKSRVTISVDT SKNQFSLKLSSVTAADTAVYYCARHDGAVAGLFDYWGQGTLVTVSSAGGGGSGG GGSGGGGSSYVLTQPPSVSVAPGQTARITCGGNNIGSKSVHWYQQPPGQAPVVV VYDDSDRPSGIPERFSGSNSGNTATLTISRVEAGDEAVYYCQVWDSSSDHVVFGG GTKLTVL (SEQ ID NO:40), or a fragment or variant thereof able to bind CD3 and BCMA having at least 70%, 75%, 80%, 85%, 90%, 95%, or 100% sequence identity to SEQ ID NO:40.

Therefore, in some embodiments, the bi-specific antibody has the amino acid sequence:
QVQLVQSGAEVKRPGASVQVSCRASGYSINTYYMQWVRQAPGAGLEWMGVINPS GVTSYAQKFQGRVTLTNDTSTNTVYMQLNSLTSADTAVYYCARWALWGDFGMDV WGKGTLVTVSSGGGGSGGGGSGGGGSDIQMTQSPSTLSASIGDRVTITCRASEGI YHWLAWYQQKPGKAPKWYKASSLASGAPSRFSGSGSGTDFTLTISSLQPDDFAT YYCQQYSNYPLTFGGGTKLEIKEPKSCDKTHTCPMADIQMTQTTSSLSASLGDRVTI SCRASQDIRNYLNWYQQKPDGTVKLLIYYTSRLHSGVPSKFSGSGSGTDYSLTISNL EQEDIATYFCQQGNTLPWTFAGGTKLEIKRAGGGSGGGSGGGSGGGSEVQLQQS GPELVKPGASMKISCKASGYSFTGYTMNWVKQSHGKNLEWMGLINPYKGVSTYNQ KFKDKATLTVDKSSSTAYMELLSLTSEDSAVYYCARSGYYGDSDWYFDVWGAGTT VTV (SEQ ID NO:41), or a fragment or variant thereof able to bind CD3 and Mesothelin having at least 70%, 75%, 80%, 85%, 90%, 95%, or 100% sequence identity to SEQ ID NO:41.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASM KISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQVQLVQSGAEVKRPGAS VQVSCRASGYSINTYYMQWVRQAPGAGLEWMGVINPSGVTSYAQKFQGRVTLTN DTSTNTVYMQLNSLTSADTAVYYCARWALWGDFGMDVWGKGTLVTVSSGGGGS GGGGSGGGGSDIQMTQSPSTLSASIGDRVTITCRASEGIYHWLAWYQQKPGKAPK LLIYKASSLASGAPSRFSGSGSGTDFTLTISSLQPDDFATYYCQQYSNYPLTFGGGT KLEIK (SEQ ID NO:42), or a fragment or variant thereof able to bind CD3 and Mesothelin having at least 70%, 75%, 80%, 85%, 90%, 95%, or 100% sequence identity to SEQ ID NO:42.

Therefore, in some embodiments, the bi-specific antibody has the amino acid sequence:
QVQLVQSGGGVVQPGRSLRLSCAASGFTFSSYGLHWVRQAPGKGLEWYAAISYD GSKKYYADSVKGRLTISRDNSKNTLYLQMNSLSPEDTALYFCARGWFVEPLSWGQ GTLVTVSSGGGGSGGGGSGGGGSQSVLTQPPSASGTPGQRVTISCSGSSSNIGS NTVNWYQQLPGTAPKLLIYSNNQRPSGVPDRFSGSKSGTSASLAISGLQSEDEADY YCAAWDDSLNGYVFGTGTKLTVLEPKSCDKTHTCPMADIQMTQTTSSLSASLGDR VTISCRASQDIRNYLNWYQQKPDGTVKLLIYYTSRLHSGVPSKFSGSGSGTDYSLTI SNLEQEDIATYFCQQGNTLPWTFAGGTKLEIKRAGGGSGGGSGGGSGGGSEVQL QQSGPELVKPGASMKISCKASGYSFTGYTMNWVKQSHGKNLEWMGLINPYKGVS TYNQKFKDKATLTVDKSSSTAYMELLSLTSEDSAVYYCARSGYYGDSDWYFDVWG AGTTVTV (SEQ ID NO:43), or a fragment or variant thereof able to bind CD3 and GPC3 having at least 70%, 75%, 80%, 85%, 90%, 95%, or 100% sequence identity to SEQ ID NO:43.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQVQLVQSGGGVVQPGR SLRLSCAASGFTFSSYGLHWVRQAPGKGLEWYAAISYDGSKKYYADSVKGRLTISR DNSKNTLYLQMNSLSPEDTALYFCARGWFVEPLSWGQGTLVTVSSGGGGSGGGG SGGGGSQSVLTQPPSASGTPGQRVTISCSGSSSNIGSNTVNWYQQLPGTAPKLLIY SNNQRPSGVPDRFSGSKSGTSASLAISGLQSEDEADYYCAAWDDSLNGYVFGTGT KLTVL (SEQ ID NO:44), or a fragment or variant thereof able to bind CD3 and GPC3 having at least 70%, 75%, 80%, 85%, 90%, 95%, or 100% sequence identity to SEQ ID NO:44.

Therefore, in some embodiments, the bi-specific antibody has the amino acid sequence:
QVQLVQSGAEVKKPGSSVRVSCKASGGTFSSYAISWVRQAPGQGLEWMGGIIPIF GTANYAQKFQGRVTITADESTSTAYMELSSLRSEDTAVYYCARDPFLHYWGQGTLV TGGGGSGGGGSGGGGSEIELTQSPLSLPVTPGEPASISCRSSQSLLHSNGYNYLD WYLQKPGQSPQLLIYLGSNRASGVPDRFSGSGSGTDFTLKISRVEAEDVGVYYCM QALQTFTFGPGTKVEIEPKSCDKTHTCPMADIQMTQTTSSLSASLGDRVTISCRASQ DIRNYLNWYQQKPDGTVKLLIYYTSRLHSGVPSKFSGSGSGTDYSLTISNLEQEDIA TYFCQQGNTLPWTFAGGTKLEIKRAGGGSGGGSGGGSGGGSEVQLQQSGPELVK PGASMKISCKASGYSFTGYTMNWVKQSHGKNLEWMGLINPYKGVSTYNQKFKDKA TLTVDKSSSTAYM ELLSLTSEDSAVYYCARSGYYGDSDWYFDVWGAGTTVTV (SEQ ID NO:45), or a fragment or variant thereof able to bind CD3 and EpCAM having at least 70%, 75%, 80%, 85%, 90%, 95%, or 100% sequence identity to SEQ ID NO:45.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQVQLVQSGAEVKKPGSS VRVSCKASGGTFSSYAISWVRQAPGQGLEWMGGIIPIFGTANYAQKFQGRVTITAD ESTSTAYMELSSLRSEDTAVYYCARDPFLHYWGQGTLVTGGGGSGGGGSGGGGS EIELTQSPLSLPVTPGEPASISCRSSQSLLHSNGYNYLDWYLQKPGQSPQLLIYLGS NRASGVPDRFSGSGSGTDFTLKISRVEAEDVGVYYCMQALQTFTFGPGTKVEI (SEQ ID NO:46), or a fragment or variant thereof able to bind CD3 and EpCAM having at least 70%, 75%, 80%, 85%, 90%, 95%, or 100% sequence identity to SEQ ID NO:46.

Therefore, in some embodiments, the bi-specific antibody has the amino acid sequence:
QVQLVESGPGVVQPGRSLRISCAVSGFSVTNYGVHWVRQPPGKGLEWLGVIWAG GITNYNSAFMSRLTISKDNSKNTVYLQMNSLRAEDTAMYYCASRGGHYGYALDYW GQGTLVTVSSGGGGSGGGGSGGGGSEIVMTQTPATLSVSAGERVTITCKASQSVS NDVTWYQQKPGQAPRLLIYSASNRYSGVPARFSGSGYGTEFTFTISSVQSEDFAVY FCQQDYSSFGQGTKLEIKREPKSCDKTHTCPMADIQMTQTTSSLSASLGDRVTISC RASQDIRNYLNWYQQKPDGTVKLLIYYTSRLHSGVPSKFSGSGSGTDYSLTISNLEQ EDIATYFCQQGNTLPWTFAGGTKLEIKRAGGGSGGGSGGGSGGGSEVQLQQSGP ELVKPGASMKISCKASGYSFTGYTMNWVKQSHGKNLEWMGLINPYKGVSTYNQKF KDKATLTVDKSSSTAYMELLSLTSEDSAVYYCARSGYYGDSDWYFDVWGAGTTVT V (SEQ ID NO:47), or a fragment or variant thereof able to bind CD3 and GD2 having at least 70%, 75%, 80%, 85%, 90%, 95%, or 100% sequence identity to SEQ ID NO:47.

Therefore, in some embodiments, the bi-specific antibody has the amino acid sequence:
MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTVEPKSCDKTHTCPQVQLVESGPGVVQPGR SLRISCAVSGFSVTNYGVHWVRQPPGKGLEWLGVIWAGGITNYNSAFMSRLTISKD NSKNTVYLQMNSLRAEDTAMYYCASRGGHYGYALDYWGQGTLVTVSSGGGGSG GGGSGGGGSEIVMTQTPATLSVSAGERVTITCKASQSVSNDVTWYQQKPGQAPRL LIYSASNRYSGVPARFSGSGYGTEFTFTISSVQSEDFAVYFCQQDYSSFGQGTKLEI KR (SEQ ID NO:48), or a fragment or variant thereof able to bind CD3 and GD2 having at least 70%, 75%, 80%, 85%, 90%, 95%, or 100% sequence identity to SEQ ID NO:48.

In some cases, the encoded bi-specific antibody further contains a signal peptide. In some embodiments, the signal peptide comprises the amino acid sequence: MDFQVQIFSFLLISASVIMSRG (SEQ ID NO:49).

Therefore, in some embodiments, the encoded bi-specific antibody has the amino acid sequence:
MDFQVQIFSFLLISASVIMSRGDIQMTQSPSSLSASVGDRVTITCRASQDVNTAVAW YQQKPGKAPKLLIYSASFLYSGVPSRFSGSRSGTDFTLTISSLQPEDFATYYCQQHY TTPPTFGQGTKVEIKRTGSTSGSGKPGSGEGSEVQLVESGGGLVQPGGSLRLSCA ASGFNIKDTYIHWVRQAPGKGLEWVARIYPTNGYTRYADSVKGRFTISADTSKNTAY LQMNSLRAEDTAVYYCSRWGGDGFYAMDVWGQGTLVTVSSEPKSCDKTHTCPM ADIQMTQTTSSLSASLGDRVTISCRASQDIRNYLNWYQQKPDGTVKLLIYYTSRLHS GVPSKFSGSGSGTDYSLTISNLEQEDIATYFCQQGNTLPWTFAGGTKLEIKRAGGG SGGGSGGGSGGGSEVQLQQSGPELVKPGASMKISCKASGYSFTGYTMNWVKQS HGKNLEWMGLINPYKGVSTYNQKFKDKATLTVDKSSSTAYMELLSLTSEDSAVYYC ARSGYYGDSDWYFDVWGAGTTVTV (SEQ ID NO:50), or a fragment or variant thereof able to bind CD3 and Her2 having at least 70%, 75%, 80%, 85%, 90%, 95%, or 100% sequence identity to SEQ ID NO:50.

Therefore, in some embodiments, the encoded bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPDIQMTQSPSSLSASVGDRVTITCRASQDVNTAVAWYQQKPGKAPKLLIYSASFL YSGVPSRFSGSRSGTDFTLTISSLQPEDFATYYCQQHYTTPPTFGQGTKVEIKRTG STSGSGKPGSGEGSEVQLVESGGGLVQPGGSLRLSCAASGFNIKDTYIHWVRQAP GKGLEWVARIYPTNGYTRYADSVKGRFTISADTSKNTAYLQMNSLRAEDTAVYYCS RWGGDGFYAMDVWGQGTLVTVSS (SEQ ID NO:51), or a fragment or variant thereof able to bind CD3 and Her2 having at least 70%, 75%, 80%, 85%, 90%, 95%, or 100% sequence identity to SEQ ID NO:51.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQIQLVQSGPELKKPGETVKISCKASGYTFTEYPIHWVKQAPGKGFKWMGMIYT DIGKPTYAEEFGRFAFSLETSASTAYLQINNLKNEDTATYFCVRDRYDSLFDYWGQ GTTLTVSSGGGGSGGGGSGGGGSDVVMTQTPLSLPVSLGDQASISCRSSQSLVH SNGNTYLHWYLQKPGQSPKWYKVSNRFSGVPDRFSGSGSGTDFTLKISRVEAED LGVYFCSQSTHVPWTFGGGTKLEIKEPKSCDKTHTCP (SEQ ID NO:52), or a fragment or variant thereof able to bind CD3 and EGFR having at least 70%, 75%, 80%, 85%, 90%, 95%, or 100% sequence identity to SEQ ID NO:52.

Therefore, in some embodiments, the bi-specific antibody has the amino acid sequence:
MDFQVQIFSFLLISASVIMSRGQIQLVQSGPELKKPGETVKISCKASGYTFTEYPIHW VKQAPGKGFKWMGMIYTDIGKPTYAEEFGRFAFSLETSASTAYLQINNLKNEDTATY FCVRDRYDSLFDYWGQGTTLTVSSGGGGSGGGGSGGGGSDVVMTQTPLSLPVSL GDQASISCRSSQSLVHSNGNTYLHWYLQKPGQSPKWYKVSNRFSGVPDRFSGS GSGTDFTLKISRVEAEDLGVYFCSQSTHVPWTFGGGTKLEIKEPKSCDKTHTCPMA DIQMTQTTSSLSASLGDRVTISCRASQDIRNYLNWYQQKPDGTVKLLIYYTSRLHSG VPSKFSGSGSGTDYSLTISNLEQEDIATYFCQQGNTLPWTFAGGTKLEIKRAGGGS GGGSGGGSGGGSEVQLQQSGPELVKPGASMKISCKASGYSFTGYTMNWVKQSH GKNLEWMGLINPYKGVSTYNQKFKDKATLTVDKSSSTAYMELLSLTSEDSAVYYCA RSGYYGDSDWYFDVWGAGTTVTV (SEQ ID NO:53), or a fragment or variant thereof able to bind CD3 and EGFR having at least 70%, 75%, 80%, 85%, 90%, 95%, or 100% sequence identity to SEQ ID NO:53.

Therefore, in some embodiments, the bi-specific antibody has the amino acid sequence:
MDFQVQIFSFLLISASVIMSRGEVQLVESGAEVKKPGDSLKISCKGSGYSFTSYWIG WVRQMPGKGLEWMGIIYPGDSETTYSPAFQGDVTISVDKSISTAYLQWNSLKASDT GIYYCARRRGNYYMDVWGNGTLVTVSSLKSGGGGSGGGGSGGGGSRSTQSALT QPASVSGSPGQSITISCTGTSSDVGGYNYVSWYQQHPGKAPKLMIYDVSNRPSGV SNRFSGSKSGNTASLTISGLQAEDEADYYCSSYTSSSTRHVFGTGTQLTVLGEPKS CDKTHTCPMADIQMTQTTSSLSASLGDRVTISCRASQDIRNYLNWYQQKPDGTVKL LIYYTSRLHSGVPSKFSGSGSGTDYSLTISNLEQEDIATYFCQQGNTLPWTFAGGTK LEIKRAGGGSGGGSGGGSGGGSEVQLQQSGPELVKPGASMKISCKASGYSFTGY TMNWVKQSHGKNLEWMGLINPYKGVSTYNQKFKDKATLTVDKSSSTAYMELLSLT SEDSAVYYCARSGYYGDSDWYFDVWGAGTTVTV (SEQ ID NO:54), or a fragment or variant thereof able to bind CD3 and CSPG4 having at least 70%, 75%, 80%, 85%, 90%, 95%, or 100% sequence identity to SEQ ID NO:54.

Therefore, in some embodiments, the bi-specific antibody has the amino acid sequence:
MFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQIQLVQSGPELKKPGETVKISCKASGYTFTEYPIHWVKQAPGKGFKWMGMIYT DIGKPTYAEEFGRFAFSLETSASTAYLQINNLKNEDTATYFCVRDRYDSLFDYWGQ GTTLTVSSGGGGSGGGGSGGGGSDVVMTQTPLSLPVSLGDQASISCRSSQSLVH SNGNTYLHWYLQKPGQSPKWYKVSNRFSGVPDRFSGSGSGTDFTLKISRVEAED LGVYFCSQSTHVPWTFGGGTKLEIKEPKSCDKTHTCP (SEQ ID NO:55), or a fragment or variant thereof able to bind CD3 and CSPG4 having at least 70%, 75%, 80%, 85%, 90%, 95%, or 100% sequence identity to SEQ ID NO:55.

Therefore, in some embodiments, the bi-specific antibody has the amino acid sequence:
MDFQVQIFSFLLISASVIMSRGQVQLQQPGAELVKPGASVKMSCKASGYTFTSYNM HWVKQTPGRGLEWIGAIYPGNGDTSYNQKFKGKATLTADKSSSTAYMQLSSLTSE DSAVYYCARSTYYGGDWYFNVWGAGTTVTVSAGGGGSGGGGSGGGGSQIVLSQ SPAILSASPGEKVTMTCRASSSVSYIHWFQQKPGSSPKPWIYATSNLASGVPVRFS GSGSGTSYSLTISRVEAEDAATYYCQQWTSNPPTFGGGTKLEIKEPKSCDKTHTCP MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTV (SEQ ID NO:56), or a fragment or variant thereof able to bind CD3 and CD20 having at least 70%, 75%, 80%, 85%, 90%, 95%, or 100% sequence identity to SEQ ID NO:56.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQVQLQQPGAELVKPGASVKMSCKASGYTFTSYNMHWVKQTPGRGLEWIGAIY PGNGDTSYNQKFKGKATLTADKSSSTAYMQLSSLTSEDSAVYYCARSTYYGGDWY FNVWGAGTTVTVSAGGGGSGGGGSGGGGSQIVLSQSPAILSASPGEKVTMTCRA SSSVSYIHWFQQKPGSSPKPWIYATSNLASGVPVRFSGSGSGTSYSLTISRVEAED AATYYCQQWTSNPPTFGGGTKLEIK (SEQ ID NO:57), or a fragment or variant thereof able to bind CD3 and CD20 having at least 70%, 75%, 80%, 85%, 90%, 95%, or 100% sequence identity to SEQ ID NO:57.

Therefore, in some embodiments, the bi-specific antibody has the amino acid sequence:
MDFQVQIFSFLLISASVIMSRGEVQLQQSGAELVKPGASVKMSCKASGYTFTSYNM HWVKQTPGQGLEWIGAIYPGNGDTSYNQKFKGKATLTADKSSSTAYMQLSSLTSE DSADYYCARSNYYGSSYWFFDVWGAGTTVTVSSGGGGSGGGGSGGGGSDIVLT QSPAILSASPGEKVTMTCRASSSVNYMDWYQKKPGSSPKPWIYATSNLASGVPAR FSGSGSGTSYSLTISRVEAEDAATYYCQQWSFNPPTFGGGTKLEIKEPKSCDKTHT CPMADIQMTQTTSSLSASLGDRVTISCRASQDIRNYLNWYQQKPDGTVKLLIYYTSR LHSGVPSKFSGSGSGTDYSLTISNLEQEDIATYFCQQGNTLPWTFAGGTKLEIKRAG GGSGGGSGGGSGGGSEVQLQQSGPELVKPGASMKISCKASGYSFTGYTMNWVK QSHGKNLEWMGLINPYKGVSTYNQKFKDKATLTVDKSSSTAYMELLSLTSEDSAVY YCARSGYYGDSDWYFDVWGAGTTVTV (SEQ ID NO:58), or a fragment or variant thereof able to bind CD3 and CD20 having at least 70%, 75%, 80%, 85%, 90%, 95%, or 100% sequence identity to SEQ ID NO:58.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPEVQLQQSGAELVKPGASVKMSCKASGYTFTSYNMHWVKQTPGQGLEWIGAIY PGNGDTSYNQKFKGKATLTADKSSSTAYMQLSSLTSEDSADYYCARSNYYGSSYW FFDVWGAGTTVTVSSGGGGSGGGGSGGGGSDIVLTQSPAILSASPGEKVTMTCRA SSSVNYMDWYQKKPGSSPKPWIYATSNLASGVPARFSGSGSGTSYSLTISRVEAE DAATYYCQQWSFNPPTFGGGTKLEIK (SEQ ID NO:59), or a fragment or variant thereof able to bind CD3 and CD20 having at least 70%, 75%, 80%, 85%, 90%, 95%, or 100% sequence identity to SEQ ID NO:59.

Therefore, in some embodiments, the bi-specific antibody has the amino acid sequence:
MDFQVQIFSFLLISASVIMSRGMARFSSSSLDLNWYSLGLQXKLSCKASGYTFTYFDI NWLRQRPEQGLEWIGVISPGDGNTNYNENFKGKATLTIDKSSTTAYIQLSRLTSEDS AVYFCARDGNFPYYAMDSWGQGTSVTVSSAKTTPKLEEGEFSEARVDIVMTQIPLS LPVILGDQASISCRSSQSLVYSNGNTYLHWFLQKPGQSPKLLIYNVSNLFSGVPDRF SGSGSGTDFTLKISRVEAEDLGIYFCSQSTHVPTFGGGTKLEIKRADAAAAGSEPKS CDKTHTCPMADIQMTQTTSSLSASLGDRVTISCRASQDIRNYLNWYQQKPDGTVKL LIYYTSRLHSGVPSKFSGSGSGTDYSLTISNLEQEDIATYFCQQGNTLPWTFAGGTK LEIKRAGGGSGGGSGGGSGGGSEVQLQQSGPELVKPGASMKISCKASGYSFTGY TMNWVKQSHGKNLEWMGLINPYKGVSTYNQKFKDKATLTVDKSSSTAYMELLSLT SEDSAVYYCARSGYYGDSDWYFDVWGAGTTVTV (SEQ ID NO:60), or a fragment or variant thereof able to bind CD3 and PSMA having at least 70%, 75%, 80%, 85%, 90%, 95%, or 100% sequence identity to SEQ ID NO:60.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPMARFSSSSLDLNWYSLGLQXKLSCKASGYTFTYFDINWLRQRPEQGLEWIGVI SPGDGNTNYNENFKGKATLTIDKSSTTAYIQLSRLTSEDSAVYFCARDGNFPYYAM DSWGQGTSVTVSSAKTTPKLEEGEFSEARVDIVMTQIPLSLPVILGDQASISCRSSQ SLVYSNGNTYLHWFLQKPGQSPKLLIYNVSNLFSGVPDRFSGSGSGTDFTLKISRVE AEDLGIYFCSQSTHVPTFGGGTKLEIKRADAAAAGS (SEQ ID NO:61), or a fragment or variant thereof able to bind CD3 and PSMA having at least 70%, 75%, 80%, 85%, 90%, 95%, or 100% sequence identity to SEQ ID NO:61.

Therefore, in some embodiments, the bi-specific antibody has the amino acid sequence:
MDFQVQIFSFLLISASVIMSRGQLQLQESGPGLVKPSETLSLTCTVSGGSISSGSYF WGWIRQPPGKGLEWIGSIYYSGITYYNPSLKSRVTISVDTSKNQFSLKLSSVTAADT AVYYCARHDGAVAGLFDYWGQGTLVTVSSAGGGGSGGGGSGGGGSSYVLTQPP SVSVAPGQTARITCGGNNIGSKSVHWYQQPPGQAPVVVVYDDSDRPSGIPERFSG SNSGNTATLTISRVEAGDEAVYYCQVWDSSSDHVVFGGGTKLTVLEPKSCDKTHT CPMADIQMTQTTSSLSASLGDRVTISCRASQDIRNYLNWYQQKPDGTVKLLIYYTSR LHSGVPSKFSGSGSGTDYSLTISNLEQEDIATYFCQQGNTLPWTFAGGTKLEIKRAG GGSGGGSGGGSGGGSEVQLQQSGPELVKPGASMKISCKASGYSFTGYTMNWVK QSHGKNLEWMGLINPYKGVSTYNQKFKDKATLTVDKSSSTAYMELLSLTSEDSAVY YCARSGYYGDSDWYFDVWGAGTTVTV (SEQ ID NO:62), or a fragment or variant thereof able to bind CD3 and BCMA having at least 70%, 75%, 80%, 85%, 90%, 95%, or 100% sequence identity to SEQ ID NO:62.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQLQLQESGPGLVKPSETLSLTCTVSGGSISSGSYFWGWIRQPPGKGLEWIGSI YYSGITYYNPSLKSRVTISVDTSKNQFSLKLSSVTAADTAVYYCARHDGAVAGLFDY WGQGTLVTVSSAGGGGSGGGGSGGGGSSYVLTQPPSVSVAPGQTARITCGGNNI GSKSVHWYQQPPGQAPVVVVYDDSDRPSGIPERFSGSNSGNTATLTISRVEAGDE AVYYCQVWDSSSDHVVFGGGTKLTVL (SEQ ID NO:63), or a fragment or variant thereof able to bind CD3 and BCMA having at least 70%, 75%, 80%, 85%, 90%, 95%, or 100% sequence identity to SEQ ID NO:63.

Therefore, in some embodiments, the bi-specific antibody has the amino acid sequence:
MDFQVQIFSFLLISASVIMSRGQVQLVQSGAEVKRPGASVQVSCRASGYSINTYYM QWVRQAPGAGLEWMGVINPSGVTSYAQKFQGRVTLTNDTSTNTVYMQLNSLTSA DTAVYYCARWALWGDFGMDVWGKGTLVTVSSGGGGSGGGGSGGGGSDIQMTQ SPSTLSASIGDRVTITCRASEGIYHWLAWYQQKPGKAPKWYKASSLASGAPSRFS GSGSGTDFTLTISSLQPDDFATYYCQQYSNYPLTFGGGTKLEIKEPKSCDKTHTCP MADIQMTQTTSSLSASLGDRVTISCRASQDIRNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTV (SEQ ID NO:64), or a fragment or variant thereof able to bind CD3 and Mesothelin having at least 70%, 75%, 80%, 85%, 90%, 95%, or 100% sequence identity to SEQ ID NO:64.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQVQLVQSGAEVKRPGASVQVSCRASGYSINTYYMQWVRQAPGAGLEWMGVI NPSGVTSYAQKFQGRVTLTNDTSTNTVYMQLNSLTSADTAVYYCARWALWGDFG MDVWGKGTLVTVSSGGGGSGGGGSGGGGSDIQMTQSPSTLSASIGDRVTITCRA SEGIYHWLAWYQQKPGKAPKWYKASSLASGAPSRFSGSGSGTDFTLTISSLQPDD FATYYCQQYSNYPLTFGGGTKLEIK (SEQ ID NO:65), or a fragment or variant thereof able to bind CD3 and Mesothelin having at least 70%, 75%, 80%, 85%, 90%, 95%, or 100% sequence identity to SEQ ID NO:65.

Therefore, in some embodiments, the bi-specific antibody has the amino acid sequence:
MDFQVQIFSFLLISASVIMSRGQVQLVQSGGGVVQPGRSLRLSCAASGFTFSSYGL HWVRQAPGKGLEWYAAISYDGSKKYYADSVKGRLTISRDNSKNTLYLQMNSLSPE DTALYFCARGWFVEPLSWGQGTLVTVSSGGGGSGGGGSGGGGSQSVLTQPPSA SGTPGQRVTISCSGSSSNIGSNTVNWYQQLPGTAPKLLIYSNNQRPSGVPDRFSGS KSGTSASLAISGLQSEDEADYYCAAWDDSLNGYVFGTGTKLTVLEPKSCDKTHTCP MADIQMTQTTSSLSASLGDRVTISCRASQDI RNYLNWYQQKPDGTVKLLIYYTSRLH SGVPSKFSGSGSGTDYSLTISNLEQEDIATYFCQQGNTLPWTFAGGTKLEIKRAGG GSGGGSGGGSGGGSEVQLQQSGPELVKPGASMKISCKASGYSFTGYTMNWVKQ SHGKNLEWMGLINPYKGVSTYNQKFKDKATLTVDKSSSTAYMELLSLTSEDSAVYY CARSGYYGDSDWYFDVWGAGTTVTV (SEQ ID NO:66), or a fragment or variant thereof able to bind CD3 and GPC3 having at least 70%, 75%, 80%, 85%, 90%, 95%, or 100% sequence identity to SEQ ID NO:66.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQVQLVQSGGGVVQPGRSLRLSCAASGFTFSSYGLHWVRQAPGKGLEWYAAI SYDGSKKYYADSVKGRLTISRDNSKNTLYLQMNSLSPEDTALYFCARGWFVEPLS WGQGTLVTVSSGGGGSGGGGSGGGGSQSVLTQPPSASGTPGQRVTISCSGSSS NIGSNTVNWYQQLPGTAPKWYSNNQRPSGVPDRFSGSKSGTSASLAISGLQSED EADYYCAAWDDSLNGYVFGTGTKLTVL (SEQ ID NO:67), or a fragment or variant thereof able to bind CD3 and GPC3 having at least 70%, 75%, 80%, 85%, 90%, 95%, or 100% sequence identity to SEQ ID NO:67.

Therefore, in some embodiments, the bi-specific antibody has the amino acid sequence:
MDFQVQIFSFLLISASVIMSRGQVQLVQSGAEVKKPGSSVRVSCKASGGTFSSYAIS WVRQAPGQGLEWMGGIIPIFGTANYAQKFQGRVTITADESTSTAYMELSSLRSEDT AVYYCARDPFLHYWGQGTLVTGGGGSGGGGSGGGGSEIELTQSPLSLPVTPGEP ASISCRSSQSLLHSNGYNYLDWYLQKPGQSPQLLIYLGSNRASGVPDRFSGSGSGT DFTLKISRVEAEDVGVYYCMQALQTFTFGPGTKVEIEPKSCDKTHTCPMADIQMTQ TTSSLSASLGDRVTISCRASQDIRNYLNWYQQKPDGTVKLLIYYTSRLHSGVPSKFS GSGSGTDYSLTISNLEQEDIATYFCQQGNTLPWTFAGGTKLEIKRAGGGSGGGSG GGSGGGSEVQLQQSGPELVKPGASMKISCKASGYSFTGYTMNWVKQSHGKNLE WMGLINPYKGVSTYNQKFKDKATLTVDKSSSTAYMELLSLTSEDSAVYYCARSGYY GDSDWYFDVWGAGTTVTV (SEQ ID NO:68), or a fragment or variant thereof able to bind CD3 and EpCAM having at least 70%, 75%, 80%, 85%, 90%, 95%, or 100% sequence identity to SEQ ID NO:68.

Therefore, in some embodiments, the bi-specific antibody has the amino acid sequence:
MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQVQLVQSGAEVKKPGSSVRVSCKASGGTFSSYAISWVRQAPGQGLEWMGGII PIFGTANYAQKFQGRVTITADESTSTAYMELSSLRSEDTAVYYCARDPFLHYWGQG TLVTGGGGSGGGGSGGGGSEIELTQSPLSLPVTPGEPASISCRSSQSLLHSNGYNY LDWYLQKPGQSPQLLIYLGSNRASGVPDRFSGSGSGTDFTLKISRVEAEDVGVYYC MQALQTFTFGPGTKVEI (SEQ ID NO:69), or a fragment or variant thereof able to bind CD3 and EpCAM having at least 70%, 75%, 80%, 85%, 90%, 95%, or 100% sequence identity to SEQ ID NO:69.

Therefore, in some embodiments, the bi-specific antibody has the amino acid sequence:
MDFQVQIFSFLLISASVIMSRGQVQLVESGPGVVQPGRSLRISCAVSGFSVTNYGVH WVRQPPGKGLEWLGVIWAGGITNYNSAFMSRLTISKDNSKNTVYLQMNSLRAEDT AMYYCASRGGHYGYALDYWGQGTLVTVSSGGGGSGGGGSGGGGSEIVMTQTPA TLSVSAGERVTITCKASQSVSNDVTWYQQKPGQAPRLLIYSASNRYSGVPARFSGS GYGTEFTFTISSVQSEDFAVYFCQQDYSSFGQGTKLEIKREPKSCDKTHTCPMADI QMTQTTSSLSASLGDRVTISCRASQDIRNYLNWYQQKPDGTVKLLIYYTSRLHSGV PSKFSGSGSGTDYSLTISNLEQEDIATYFCQQGNTLPWTFAGGTKLEIKRAGGGSG GGSGGGSGGGSEVQLQQSGPELVKPGASMKISCKASGYSFTGYTMNWVKQSHG KNLEWMGLINPYKGVSTYNQKFKDKATLTVDKSSSTAYMELLSLTSEDSAVYYCAR SGYYGDSDWYFDVWGAGTTVTV (SEQ ID NO:70), or a fragment or variant thereof able to bind CD3 and GD2 having at least 70%, 75%, 80%, 85%, 90%, 95%, or 100% sequence identity to SEQ ID NO:70.

Therefore, in some embodiments, the bi-specific antibody has the amino acid sequence:

MDFQVQIFSFLLISASVIMSRGMADIQMTQTTSSLSASLGDRVTISCRASQDIRNYLN WYQQKPDGTVKLLIYYTSRLHSGVPSKFSGSGSGTDYSLTISNLEQEDIATYFCQQ GNTLPWTFAGGTKLEIKRAGGGSGGGSGGGSGGGSEVQLQQSGPELVKPGASMK ISCKASGYSFTGYTMNWVKQSHGKNLEWMGLINPYKGVSTYNQKFKDKATLTVDK SSSTAYMELLSLTSEDSAVYYCARSGYYGDSDWYFDVWGAGTTVTVEPKSCDKTH TCPQVQLVESGPGVVQPGRSLRISCAVSGFSVTNYGVHWVRQPPGKGLEWLGVI WAGGITNYNSAFMSRLTISKDNSKNTVYLQMNSLRAEDTAMYYCASRGGHYGYAL DYWGQGTLVTVSSGGGGSGGGGSGGGGSEIVMTQTPATLSVSAGERVTITCKAS QSVSNDVTWYQQKPGQAPRLLIYSASNRYSGVPARFSGSGYGTEFTFTISSVQSED FAVYFCQQDYSSFGQGTKLEIKR (SEQ ID NO:71), or a fragment or variant thereof able to bind CD3 and GD2 having at least 70%, 75%, 80%, 85%, 90%, 95%, or 100% sequence identity to SEQ ID NO:71.

Candidate engineered antibodies for inclusion in the fusion polypeptides, or the fusion polypeptides themselves, may be screened for activity using a variety of known assays. For example, screening assays to determine binding specificity are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6.

In some embodiments, the bispecific antibody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Bispecific antibodies which have been altered will therefore remain administrable for a longer period of time with reduced or no immune response-related side effects than corresponding bispecific antibodies which have not undergone any such alteration(s). One of ordinary skill in the art will understand how to determine whether, and to what degree an antibody must be altered in order to prevent it from eliciting an unwanted host immune response.

### Pharmaceutical Composition

Also disclosed is a pharmaceutical composition comprising a disclosed molecule in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutically acceptable carriers include any and all suitable solvents, dispersion media, coatings, antibacterial and antifungal agents, isotonicity agents, antioxidants and absorption delaying agents, and the like that are physiologically compatible with a bispecific antibody of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

### Methods of Treatment

Adoptive transfer of the disclosed off-the-shelf T cells can be used to treat a variety of diseases and conditions in an allogeneic subject, such as cancer, autoimmune disease, diabetes, neurological disorders, chronic viral infections, bacterial infections, parasitic infections, Alzheimer's disease, heart disease. T disclosed off-the-shelf T cells may also be genetically engineered to express one or more CARs.

The disclosed off-the-shelf T cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed off-the-shelf T cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In some embodiments, the method is used to treat a cancer in the allogeneic subject. In some aspects, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed off-the-shelf T cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed off-the-shelf T cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (M DX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed off-the-shelf T cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with off-the-shelf T cells for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD4OL, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination off-the-shelf T cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with off-the-shelf T cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with off-the-shelf T cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed off-the-shelf T cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed off-the-shelf T cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

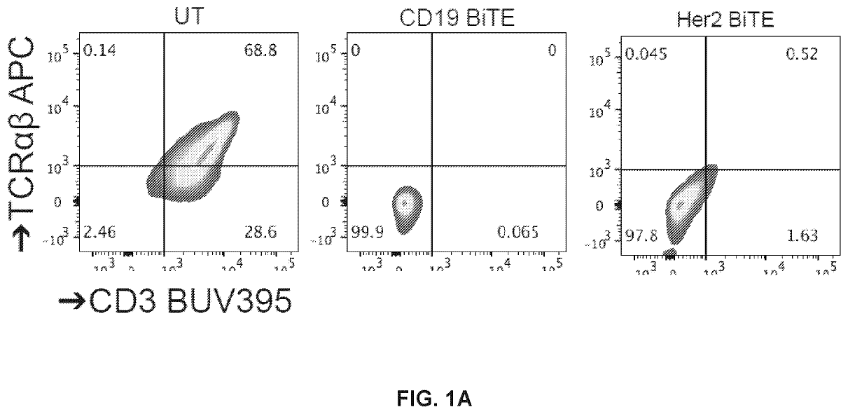

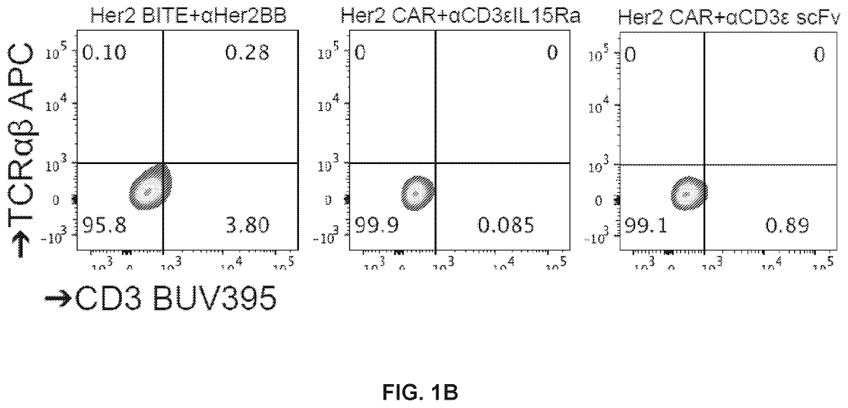

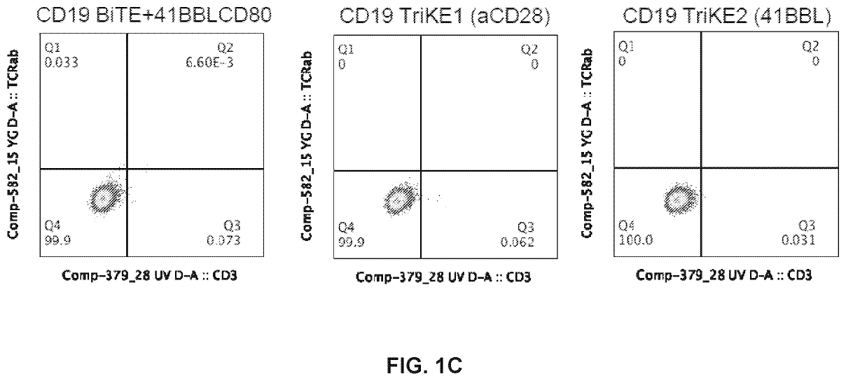

FIGS. 1A to 1C show CD3/TCRαβ expression in vitro after treatment with a CD19 BiTE or Her2 BiTE (FIG. 1A); Her2 BITE+αHer2BB, Her2 CAR+αCD3ϵIL15RA, or Her2 CAR+αCD3ϵ scFV (FIG. 1B); and CD19 BiTE+41BBLCD80, CD19 TriKE1 (aCD28), or CD19 TriKE2 (41BBL) (FIG. 1C).

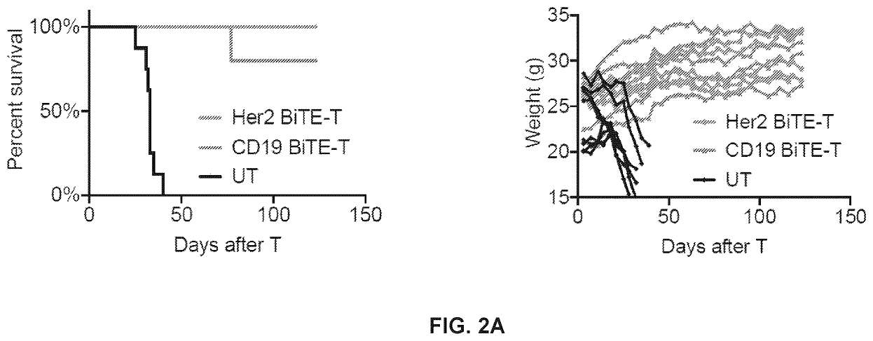

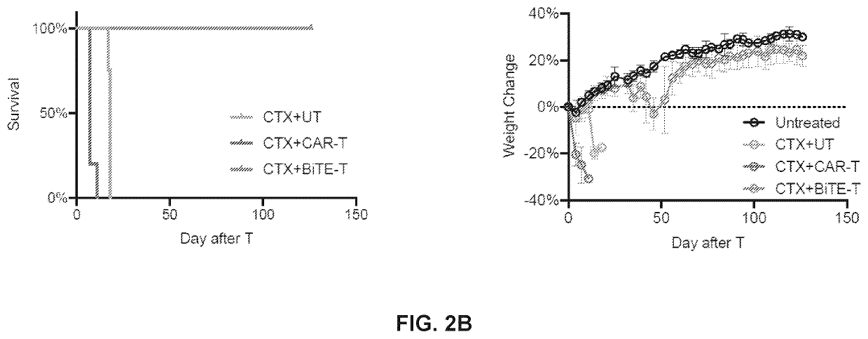

FIGS. 2A and 2B show GvDH model test. FIG. 2A shows NSG mice treated with whole body irradiation and 1e7 Her2 BiTE-T or CD19 BiTE-T cells. FIG. 2B shows NSG mice treated with chemotherapy and 1e7 CTX+CAR-T or CTX+BiTE-T cells.

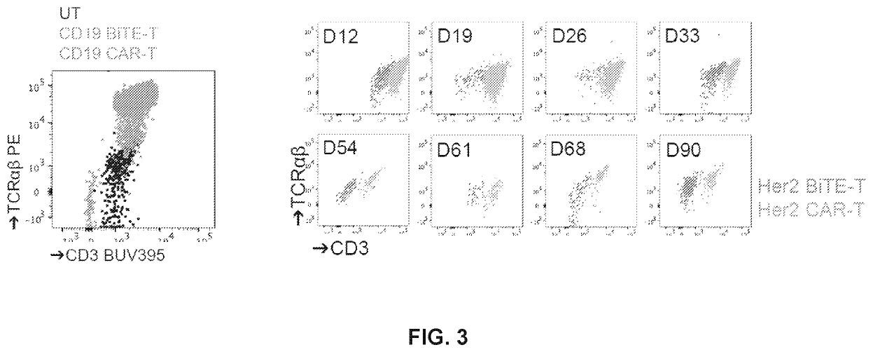

FIG. 3 shows CD3/TCRαβ expression in vivo without antigen. NSG mice received irradiation and then T cells. Donor T cells in peripheral blood were evaluated at certain time points. D12, Day 12 after T cell injection.

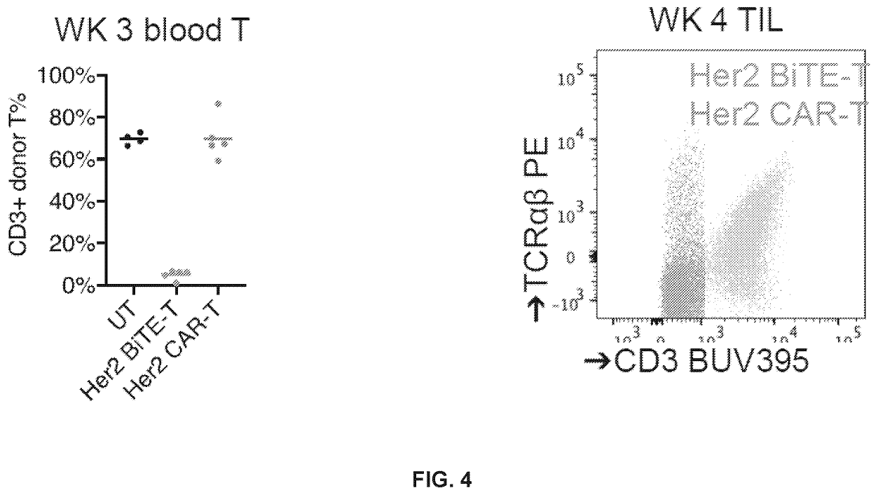

FIG. 4 shows CD3/TCRαβ expression in vivo with antigen. NSG mice were injected with tumor cells and then received T cell treatment. Donor T cells in peripheral blood or tumor were evaluated at certain time points.

### Example 2

Next, in vitro serial killing assays were set up to evaluate TCRαβ and CD3 on BiTE-T cells in the presence of target antigen. T cells were repeatedly challenged with target cells until they failed to kill. TCRαβ and CD3ϵ expression were evaluated every 48 hr during this period. For both CD19 and Her2 targets, BiTE-T cells maintained low TCRαβ/CD3 through the challenge assay in contrast to CAR-T and UT cells. In vivo, Her2-expressing solid tumor model was used a to evaluate Her2 BiTE-T cells. NSG mice were first inoculated with Her2-expressing human melanoma cell A375 (A375.Her2) and then received BiTE-T or UT treatment. Blood T cells and tumor infiltrating T cells were evaluated by flow cytometry. In peripheral blood, Her2 BiTE-T cells showed limited presence with low CD3ϵ. Tumor infiltrated Her2 BiTE-T cells also show lower TCRαβ/CD3 than CAR-T cells. These results indicate that BiTE-T cells maintain low CD3ϵ and TCRαβ in the presence of target antigen.

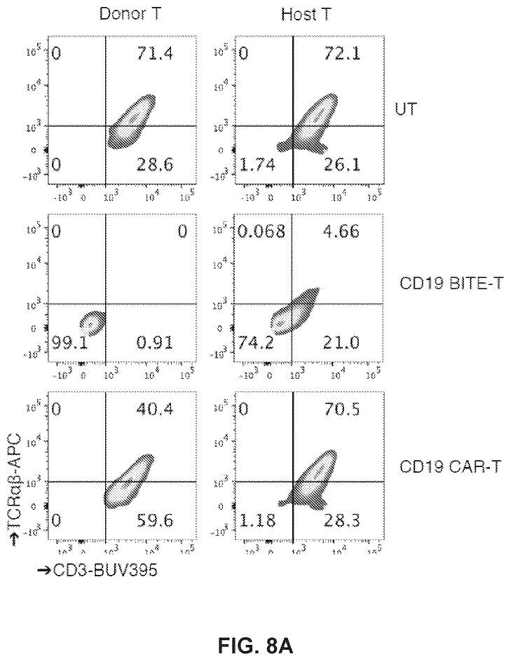

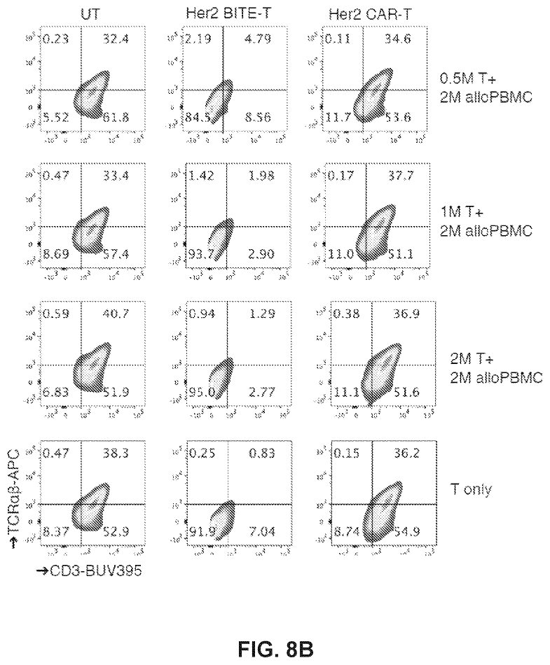

Allogeneic OTS T cells are expected to overcome host rejection in order to persist longer and maximize their efficacy. One important mechanism of host rejection is that host T cells recognize non-self antigens through TCR. CD19 BiTE-T cells with were co-cultured HLA mismatched T cells for 72 hr and found BiTE-T reduced CD3 and TCRαβ on recipient T cells. Similar results were also observed when alloMLR assays were set up by co-culturing CD19 or Her2 BiTE-T cells with HLA mismatched PBMCs (FIG. 8). These data suggest that donor BiTE-T cells have potential to overcome host rejection by releasing BiTEs binding TCRαβ/CD3 on host T cells.

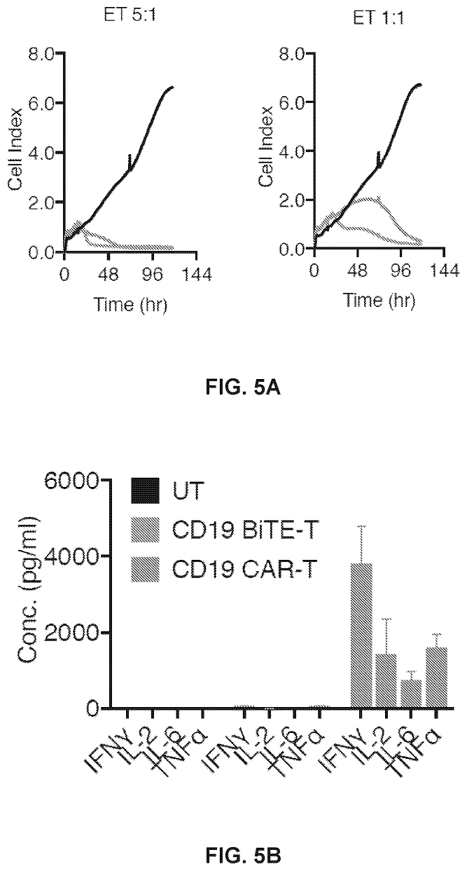

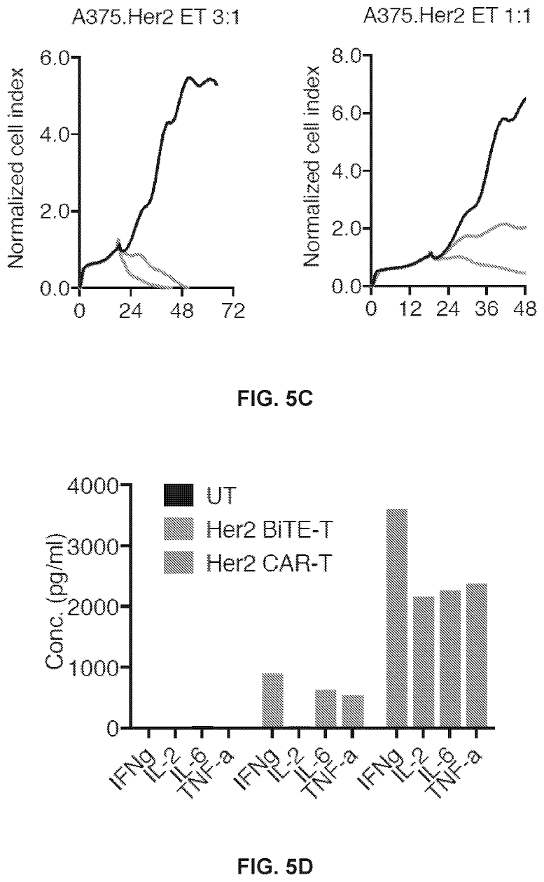

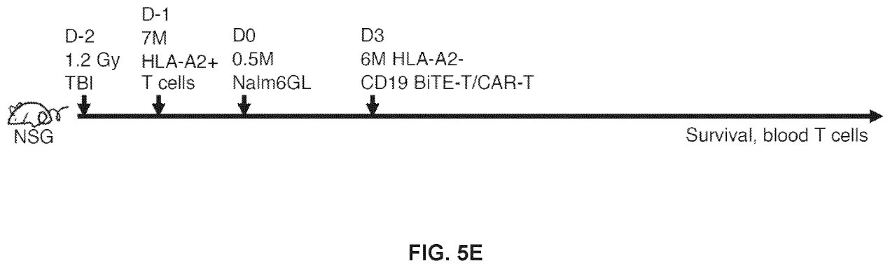

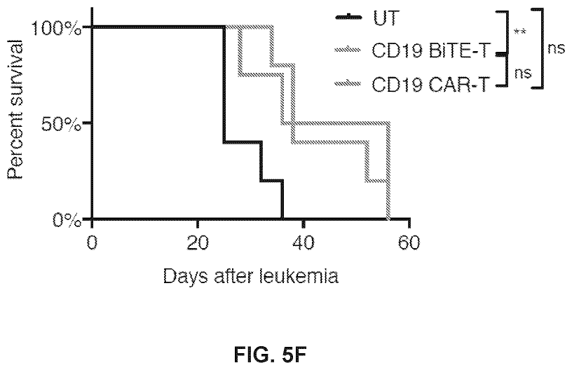

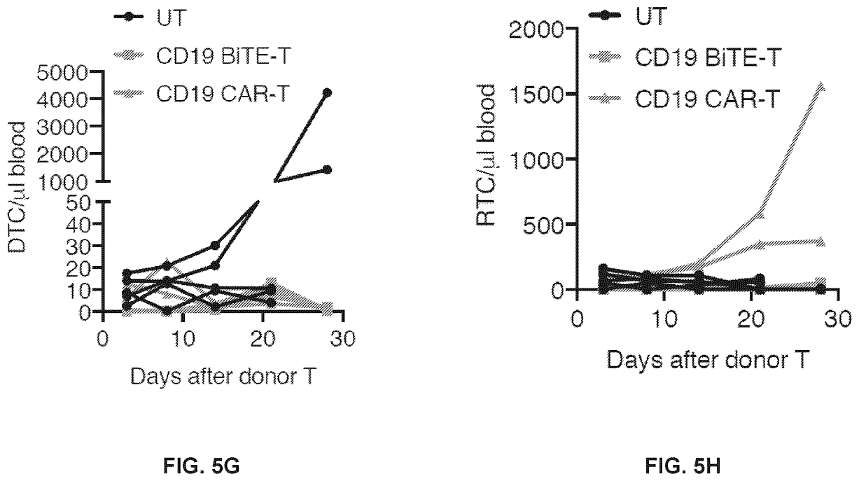

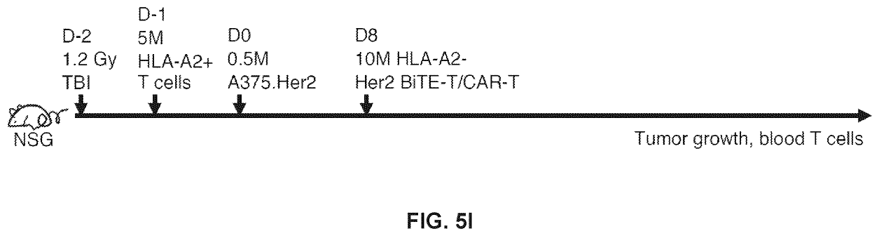

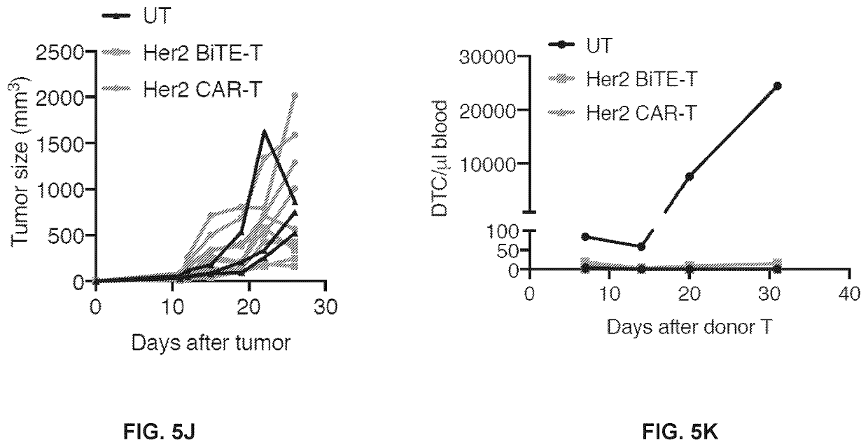

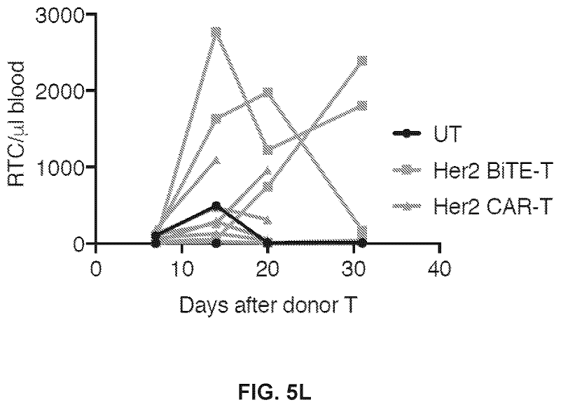

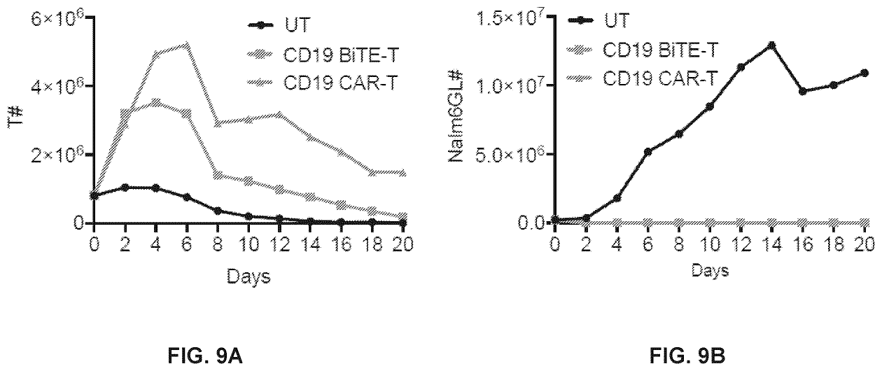

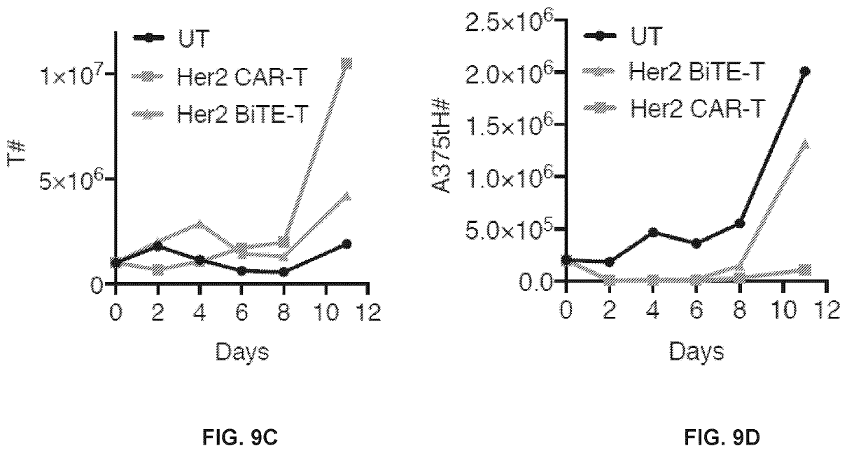

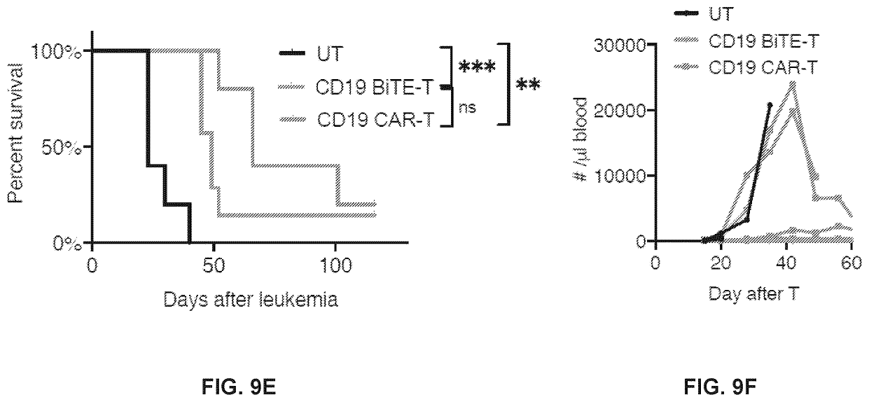

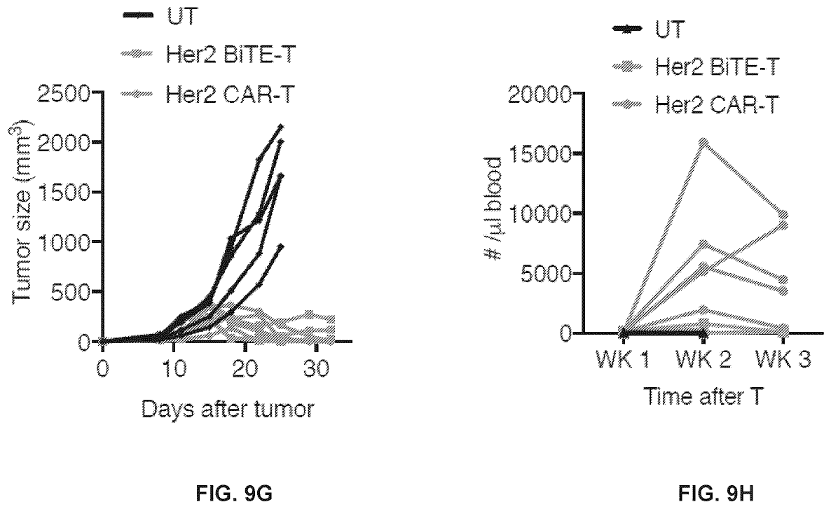

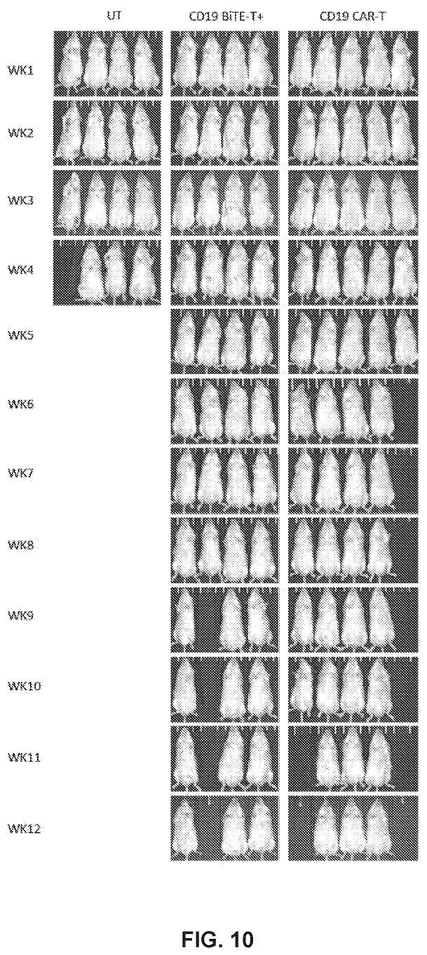

The anti-cancer activities of BiTE-T cells have been demonstrated by other groups (Iwahori, K. et al. Mol Ther 2015 23:171-178; Choi, B.D. et al. Nat Biotechnol 2019 37:1049-1058; Liu, X. et al. Protein Cell 2017 8:514-526). Here side-by-side comparisons were conducted between BiTE-T and the second-generation CAR-T cells on their anti-cancer activities. CD19 or Her2 BiTE-T and CAR-T cells sharing the same scFv were evaluated. For both targets, BiTE-T cells show comparable, if not better, killing than CAR-T cells in vitro (FIG. 5A, 5C). BiTE-T cells produced only a fraction of cytokines that CAR-T cell produced (FIG. 5B, 5D), including IFNγ, IL2, IL6, and TNFα. This suggests that BiTE-T cells may be safer than CAR-T cells. However, CAR-T cells outperformed BiTE-T cells in serial killing assays (FIG. 9A-9D), and also offered better survival, persistence, and efficacy in NSG models transplanted with NALM6 or A375.Her2 cells (FIG. 9E-9H). This is not surprising since second generation CAR-T cells have built-in costimulatory domains.

One potential advantage of BiTE-T cells is the capability of mobilizing bystander or host T cells. Thus, a mouse model was adopted to mimic an environment with pre-existing host T cells (Mo, F. et al. Nat Biotechnol 2021 39:56-63). NSG mice were irradiated and then engrafted with activated HLA-A2+ human T cells. Mice were then injected with tumor cells and later treated with HLA-A2− BiTE-T or CAR-T cells later (FIG. 5E). In the NSG NALM6 model with pre-existing T cells, both CD19-targeting BiTE-T and CAR-T cells showed a comparable survival benefit over UT cells (FIG. 5F). Donor CD19 CAR-T cell expansion was also curbed in this model although host T cells showed expansion in the CAR-T treated group (FIG. 5G, 5 H). In the Her2+tumor model with pre-existing host T cells (FIG. 5I), CAR-T cells were not able to inhibit tumor growth while BiTE-T cells still could (FIG. 5J). In peripheral blood, both Her2-targeting BiTE-T and CAR-T cells showed very limited persistence (FIG. 5K). However, Her2 BiTE-T cells induced greater host T cell expansion than CAR-T cells. (FIG. 5L).

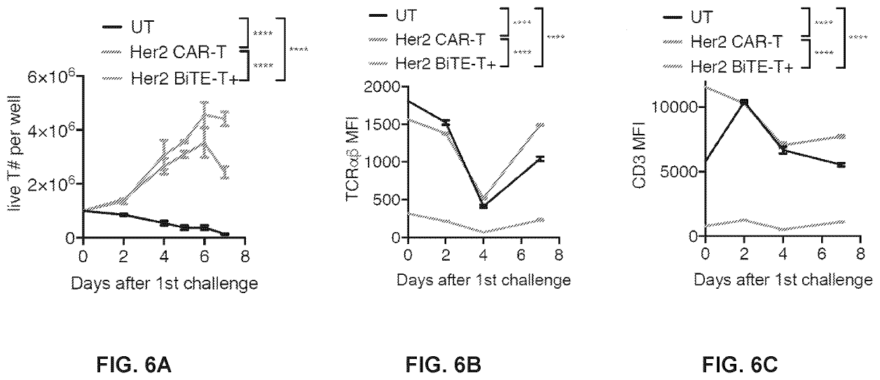

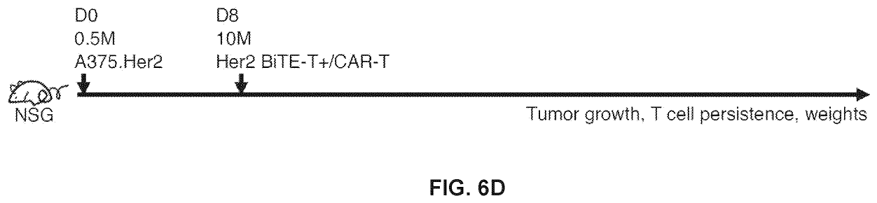

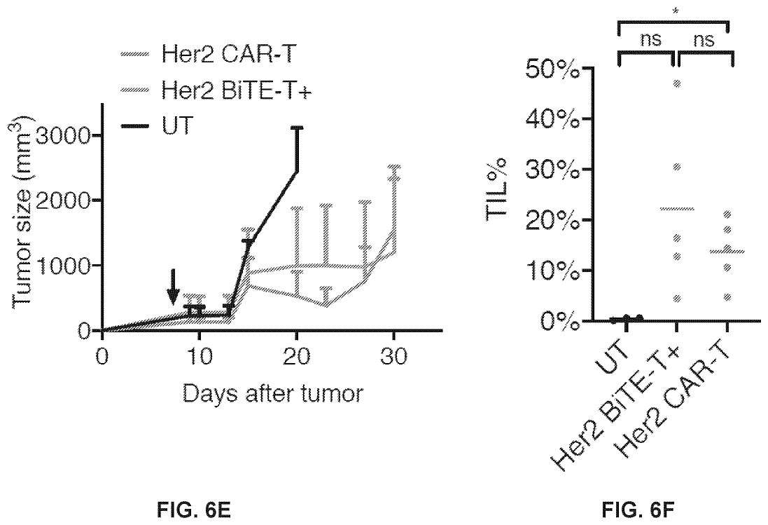

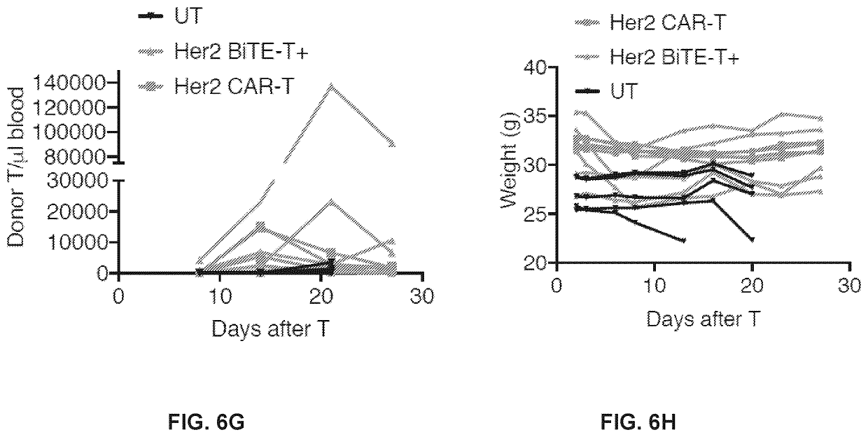

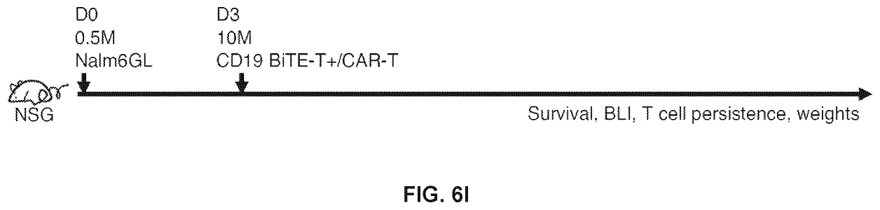

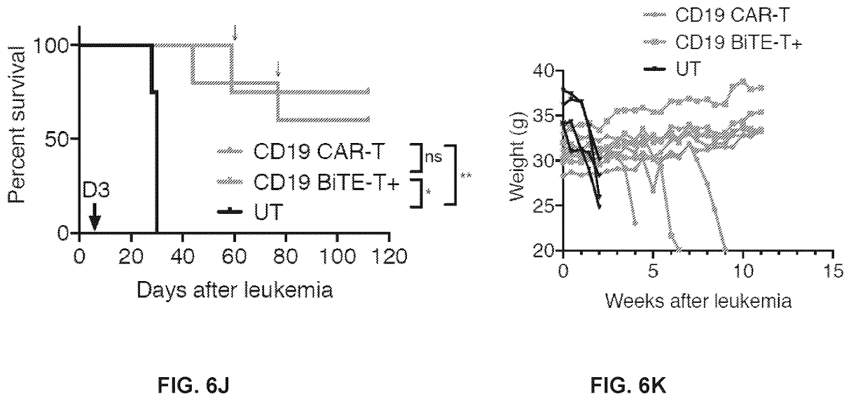

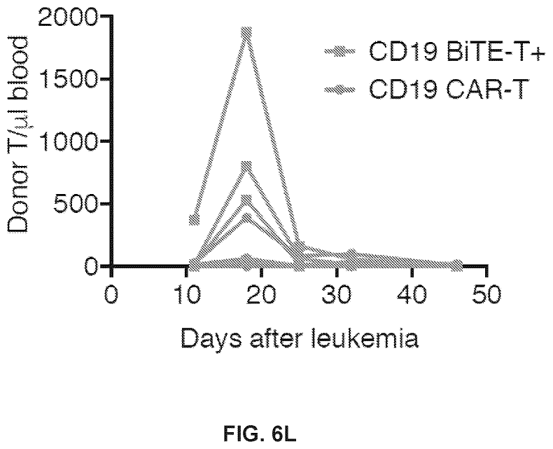

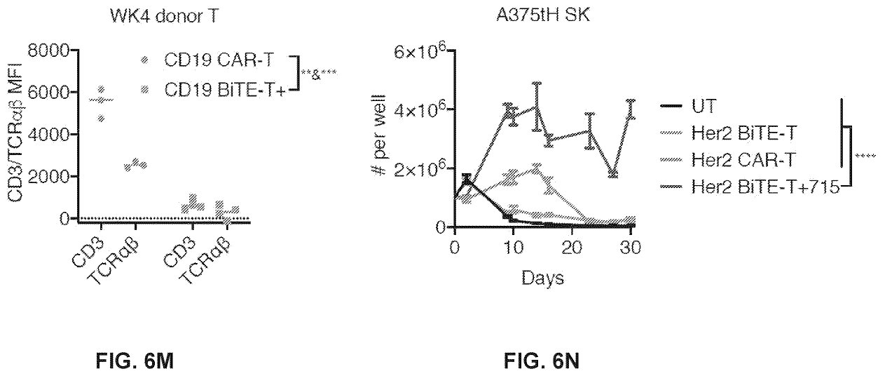

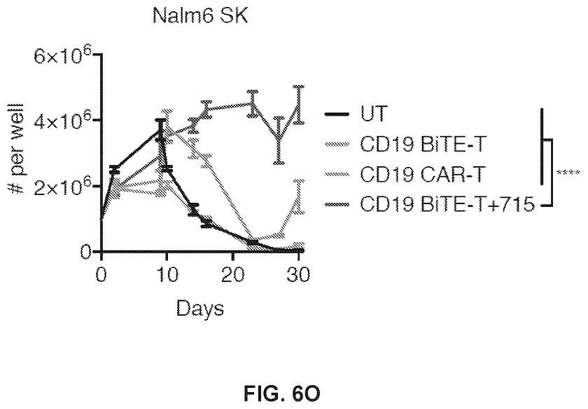

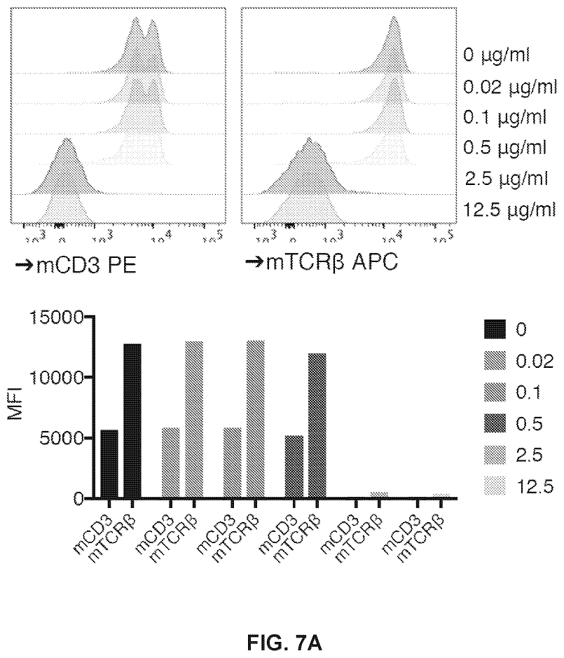

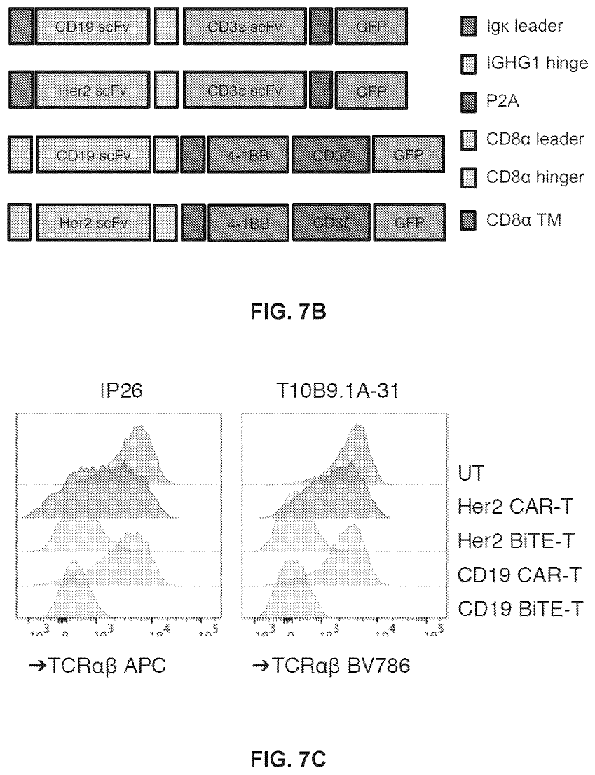

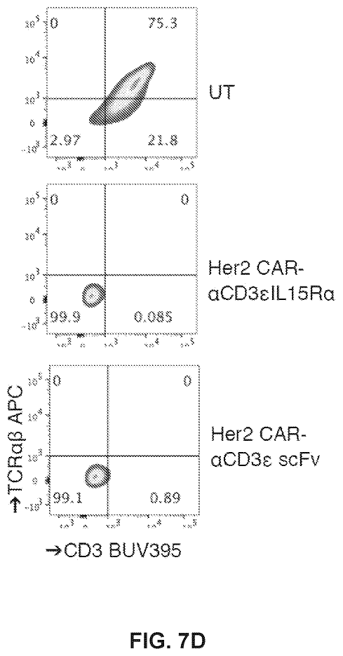

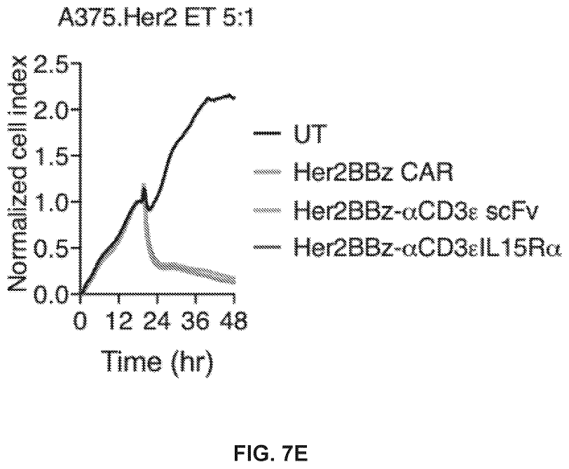

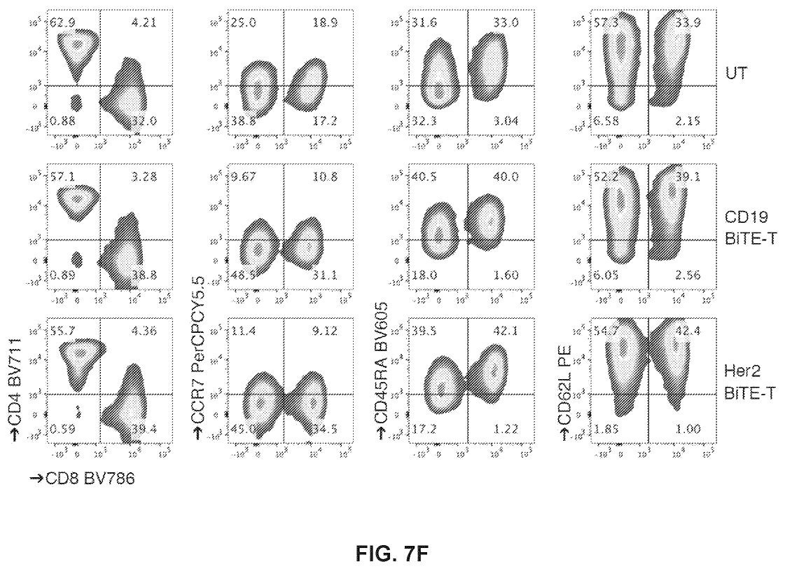

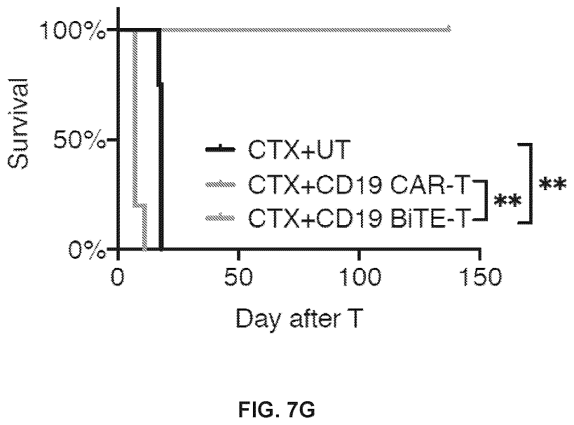

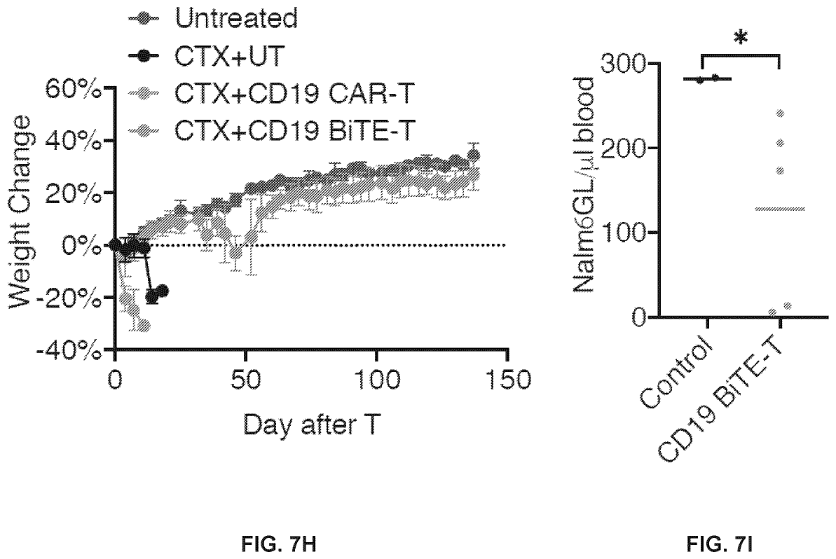

To enhance efficacy, co-stimulation was added to BiTE-T cells. T cells were co-transduced with BiTE and a combo of 4-1BBL and CD80 (Velasquez, M.P. et al. Cancer Immunol Res 2017 5:860-870) to make the next generation BiTE-T cells. These enhanced Her2 BiTE-T cells show better proliferation than Her2 CAR-T cells in serial killing assay while maintaining low levels of CD3/TCRαβ (FIG. 6A-6C). In vivo (FIG. 6D), Her2 BiTE-T cells show comparable tumor suppression, blood persistence, tumor infiltration and weights to CAR-T cells (FIG. 6E-6H). Similarly, in the NALM6 model (FIG. 61), the enhanced CD19 BiTE-T cells have comparable efficacy and possibly better blood persistence than CAR-T cells (FIG. 6J-6L, 10). Enhanced CD19 BiTE-T cells maintained low CD3/TCRαβ (FIG. 6M) in vivo. BiTE-T cells were also enhanced by engineering with a combo of IL7 and IL15 genes. BiTE T cells engineered with cytokines outperformed CAR-T cells in serial killing assay in vitro (FIG. 6N, 6O).

This study proposes a new strategy for allogeneic OTS T cell therapy. CAR-T cell therapy is currently one of the most powerful cancer therapeutics approved by FDA. Recent clinical trials have shown that allogeneic CAR-T cells could potentially replace their autologous counterpart although with some sacrifice of efficacy. BiTE-T cells may have some advantages over allogeneic OTS CAR-T cells. First, BiTE-T cells are less complex to produce and are a naturally “pure” product. BiTE-T cell production is essentially the same as autologous CAR-T production while allogeneic CAR-T cells require gene editing and TCRαβ negative purification. Unlike current CAR-T products having “dormant” untransduced T cells, BiTE-T cell products will be fully equipped with BiTEs whether transduced or not, so the final product will have 100% activity. Second, BiTE-T cells have the potential to overcome host rejection without lymphodepletion or further engineering. Current allogeneic CAR-T cells require a harsh lymphodepletion regimen for a longer therapeutic window or more engineering such as an alloimmune defense receptor (Mo, F. et al. Nat Biotechnol 2021 39:56-63) to kill host T cells. In contrast, BiTE-T cells have the potential to expand and re-target host T cells thus possibly requiring no or light lymphodepletion while offering extra killing. Third, BiTE-T cells are safer as they release less cytokines when killing targets. One concern about BiTE-T cell products without TCRαβ negative purification is that the contaminated untransduced T cells can potentially cause GvHD. However, high doses of human BiTE-T cells with reasonable untransduced T cell contamination did not induce GvHD in immune deficient NSG mouse model. If used in the setting of no or low lymphodepletion, the risk of BiTE-T causing GvHD would be even lower. Another potential disadvantage of BiTE-T cells is that they are less potent than CAR-T cells. This can largely be fixed by adding co-stimulation or cytokines.

In summary, BiTE-T cells provide a novel and simple way for “off-the-shelf” allogeneic T cell therapy warranting further evaluation in clinical trials.

### Methods

### Study Design

The purpose of this study was to develop a novel allogeneic “off-the-shelf” T cell therapy. CD19- and Her2-targeting BiTE engineered T cells were evaluated for CD3/TCRαβ expression. GvHD risk of BiTE-T cells was evaluated using chemo-conditioned or whole-body irradiated NSG mice. Side-by-side comparisons were also performed between BiTE-T and CAR-T cells on cytotoxicity, cytokines, serial killing, and in vivo efficacy with or without host T cells. Two xenograft mouse models were used to compare efficacy between BiTE-T and CAR-T cells, including hematological and solid tumor. A variety of healthy donors were used to produce engineered T cells. All experiments were independently repeated at least twice.

### Cells and Medium

The NALM6GL cell line expressing both GFP and firefly luciferase was purchased from ATCC and cultured in RPM11640 medium supplemented with 10% FBS, L-glutamine and Pen/Strep (RPMI10). The human melanoma A375 cell line was a gift from Dr. Cecilia Ramello and cultured in DM EM medium supplemented with 10% FBS, L-glutamine and Pen/Strep. Her2-expressing A375 (A375.Her2) were made by transducing A375 cells using retrovirus expressing a truncated Her2. Human T cells were cultured in RPMI10 supplemented with 10 ng/ml IL7 and 5 ng/ml IL15.

Retroviral Constructs, γ-Retrovirus Production and T Cell Transduction DNAs of human Her2 CAR, human CD19 BiTE, Her2 BiTE, 4-1BBL-CD80, and other constructs were synthesized and subcloned to an SFG retroviral vector by Genewiz (South Plainfield, NJ). Human CD19 CAR has been described previously (Li, G. et al. JCI Insight 2018 3). Her2 CAR and Her2 BiTE use the same scFv derived from Trastuzumab. CD19 CAR and CD19 BiTE use the same scFv derived from FMC63 clone. Recombinant γ-retrovirus production has been described previously (Li, G., et al. Methods Mol Biol 2017 1514:111-118). Briefly, retroviral constructs containing CAR or BiTE were transiently transfected to H29 cells. Supernatant from transfected H29 were used to transduce RD114 cells to make a stable producer cell line. Retroviral supernatant from RD114 cells were harvested, 0.45 μm filtered, and cryopreserved for future use. For T cell transduction, healthy donor leukopaks were purchased from Stemcell Technologies (Vancouver, Canada) and ALLCELLS (Alameda, CA). PBMCs were isolated using standard Ficoll method and cryopreserved for future use. At day 0, T cells were isolated from the cryopreserved PBMCs using a human T cell isolation kit (Stemcell Technologies). T cells were then activated using human CD3/CD28 dynabeads (ThermoFisher Scientific, Waltham, MA). At day 1 and day 2, T cells were transduced using fresh BiTE or CAR retrovirus in RetroNectin (Takara Bio, Mountain View, CA) coated plates at 2000 g, 32° C. for one hour. At day 3 fresh medium with cytokines were added. Dynabeads were removed at day 6 and T cells were further expanded in complete medium with ng/ml human IL7 and 5 ng/ml human IL15. Medium and cytokines were replenished every 2-3 days. Day 8-14 T cells were used for in vivo study.

### Flow Cytometry

The following antibody clones were used for flow cytometry. From BD: CD3 (Clone SK7), CD8 (Clone RPA-T8), CD45 (Clone H130), TCRαβ (Clone T10B9.1A-31), CD45RA (HI100), PD1 (Clone EH12.2H7), CD69 (Clone FN50), CD25 (Clone 2A3). From Biolegend: CD4 (Clone OKT4), TCRαβ (Clone IP26), CCR7 (Clone G043H7), CD62L (Clone DREG-56). Fc receptor binding inhibitor (ThermoFisher) were routinely used for staining. Whole blood samples were stained and lysed using BD FACS lysing solution. CountBright absolute counting beads (ThermoFisher) were added for measuring cell numbers. Flow data were acquired on BD LSRII or BD FACSymphony flow cytometer. Data were analyzed using FlowJo Version 10.

### ELISA

An Ella machine (ProteinSimple, San Jose) was used for ELISA. Twenty-four to 72 hr supernatant from cytotoxicity or TCR stimulation assay were added to ELLA cartridges for cytokine measurements.

### TCR/CD3 Stimulation Assay

For human T cell stimulation assay, non-tissue culture treated 24-well plates were coated with 1 μg/ml human TCRαβ antibody (Clone I P26) overnight. T cells were added and cultured in the plates. For activation marker, T cells were subjected to flow analysis after 5-hr stimulation. Supernatant were collected after 24-hr stimulation and cytokines were measured by ELISA. For BiTE stimulation, human CD19 BiTE (Blinatumomab) was purchased from BPS Bioscience (San Diego, CA). T cells were cocultured with 3T3.hCD19 or 3T3.mCD19 cells with increasing Blinatumomab concentrations. For mouse T cell stimulation, T cells were isolated from spleen and added to 24 well plates coated with mouse CD3ϵ antibody (Clone 145-2C11). After 5 hr, cells were analyzed by flow cytometry.

### Cytotoxicity and Serial Killing Assay

An xCELLigence RTCA instrument (Agilent Technologies, Santa Clara, CA) was used for cytotoxicity assay. T cells were co-cultured with target cells at various E:T ratios in RTCA E-plates and real-time cell killing was recorded on the instrument.

For serial killing assays, T cells were co-cultured with target cells at a 5:1 E:T ratio in non-tissue culture treated 24-well plates. Every two days or longer a small fraction of T cells were analyzed by flow cytometry for phenotype and cell counts while the remaining cells were transferred to a new plate with fresh medium and target cell challenge.

### Mice Study

All mice protocols were approved by the Institutional Animal Care and Use Committee of the University of South Florida. NSG mice were purchased from the Jackson Lab and bred in the USF animal facility. Eight-to-twelve weeks old male NSG mice were used for in vivo studies. For GvHD evaluation, NSG mice underwent 2.5 Gy whole body irradiation or 250 mg/kg cyclophosphamide (CTX) then received T cells at ap 10-million dose the following day. Mice were monitored for survival and any signs of GvHD (hunched posture, slow mobility, weight loss etc.). Mice having more than 20% weight loss and other GvHD symptoms were sacrificed. For efficacy study on hematological tumor model, NSG mice were intravenously injected with 0.5 million NALM6GL cells. Three days later, mice were intravenously injected with million

CAR-T, BiTE-T or UT cells. Survival was monitored twice a week. For the solid tumor model, NSG mice were subcutaneously injected with 0.5 million A375.Her2 cells on the right flank. Seven to ten days later mice were intravenously injected with million CAR-T, BiTE-T or UT cells. Blood was collected weekly and tumor tissues collected at end-point. For mice with pre-existing T cells, NSG mice received 1.2 Gy whole body irradiation then were subsequently engrafted with activated T cells. Mice were injected with tumor cells and treated with HLA mismatched T cells. For details, see the timelines in the figures. Tumor tissues were processed on a gentIMACS Octo Dissociator (Miltenyi Biotec, Bergisch Gladbach, Germany) and digested with Liberase and DNase I (Roche, Mannheim, Germany). Isolated Tumor cells were subjected to flow analysis.

### Statistical Methods.

GraphPad Prism 8 was used for statistical analysis as indicated in figure legends. Data were presented as means±s.d. Unpaired parametric t test was used for 2-group comparison. One-way ANOVA was used for multiple-group comparison. Survival was compared using log-rank tests.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating cancer in a subject, comprising obtaining immune effector cells from an allogeneic donor;
engineering the immune effector cells to express an anti-CD3 multi-specific antibody, wherein the antibody is configured to bind a CD3 complex on the immune effector cells and a second antigen on another cell in a manner sufficient to activate the CD3 complex; and
administering the engineered immune effector cell to the subject in an amount effective to treat the cancer.

1. The method of claim 1, wherein the antibody is a bi-specific antibody.

2. The method of claim 1, wherein the second antigen is a tumor antigen.

3. The method of claim 1, wherein the second antigen is selected from EpCAM, CCR5, CD19, HER-2 neu, HER-3, HER-4, EGFR, PSMA, CEA, MUC-1 (mucin), MUC2, MUC3, MUC4, MUC5_{A}c, MUC5_{B}, MUC7, DhCG, Lewis-Y, CD20, CD33, CD30, ganglioside GD3, 9-0-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRvIII, LG, SAS, and CD63.

4. The method of any one of claims 1 to 4, wherein the immune effector cells are further engineered to express a chimeric antigen receptor (CAR).

5. The method of any one of claims 1 to 5, wherein the immune effector cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

6. A method for enhancing CAR-T cells for allogeneic cell transfer, comprising engineering the CAR-T cells to secrete a monospecific anti-CD3 antibody.

7. The method of claim 7, wherein the monospecific anti-CD3 antibody is a single chain variable fragment (scFv).

8. A CAR-T cell engineered to express a monospecific anti-CD3 antibody.

9. A method for enhancing immune effector cells for allogeneic cell transfer, comprising
engineering the immune effector cells to express a membrane bound anti-CD3 antibody,
wherein the anti-CD3 antibody is configured to bind a CD3 complex on the immune effector cells and in a manner sufficient to auto-activate the CD3 complex.


SEQUENCE LISTING (71 sequences)

SEQ ID NO: 1 - protein, 111 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRA

SEQ ID NO: 2 - protein, 120 aa, Artificial Sequence
  Synthetic Construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 3 - protein, 132 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEPKSCDKTHTCP

SEQ ID NO: 4 - protein, 107 aa, Artificial Sequence [sequence omitted]

SEQ ID NO: 5 - protein, 117 aa, Artificial Sequence
  Synthetic Construct
QIQLVQSGPELKKPGETVKISCKASGYTFTEYPIHWVKQAPGKGFKWMGMIYTDIGKPTYAEEFGRFAFSLETSASTAYLQINNLKNEDTATYFCVRDRYDSLFDYWGQGTTLTVSS

SEQ ID NO: 6 - protein, 112 aa, Artificial Sequence
  Synthetic Construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 7 - protein, 121 aa, Artificial Sequence
  Synthetic Construct
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKS

SEQ ID NO: 8 - protein, 115 aa, Artificial Sequence
  Synthetic Construct
RSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 9 - protein, 121 aa, Artificial Sequence [sequence omitted]

SEQ ID NO: 10 - protein, 106 aa, Artificial Sequence [sequence omitted]

SEQ ID NO: 11 - protein, 122 aa, Artificial Sequence
  Synthetic Construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 12 - protein, 106 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 13 - protein, 129 aa, Artificial Sequence
  Synthetic Construct
  misc_feature (20)..(20): Xaa can be any naturally occurring amino acid
MARFSSSSLDLNWYSLGLQXKLSCKASGYTFTYFDINWLRQRPEQGLEWIGVISPGDGNTNYNENFKGKATLTIDKSSTTAYIQLSRLTSEDSAVYFCARDGNFPYYAMDSWGQGTSVTVSSAKTTPKL

SEQ ID NO: 14 - protein, 120 aa, Artificial Sequence
  Synthetic Construct
DIVMTQIPLSLPVILGDQASISCRSSQSLVYSNGNTYLHWFLQKPGQSPKLLIYNVSNLFSGVPDRFSGSGSGTDFTLKISRVEAEDLGIYFCSQSTHVPTFGGGTKLEIKRADAAAAGS

SEQ ID NO: 15 - protein, 122 aa, Artificial Sequence
  Synthetic Construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSA

SEQ ID NO: 16 - protein, 107 aa, Artificial Sequence
  Synthetic Construct
YVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 17 - protein, 119 aa, Artificial Sequence
  Synthetic Construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSS

SEQ ID NO: 18 - protein, 107 aa, Artificial Sequence
  Synthetic Construct
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 19 - protein, 117 aa, Artificial Sequence
  Synthetic Construct
QVQLVQSGGGVVQPGRSLRLSCAASGFTFSSYGLHWVRQAPGKGLEWYAAISYDGSKKYYADSVKGRLTISRDNSKNTLYLQMNSLSPEDTALYFCARGWFVEPLSWGQGTLVTVSS

SEQ ID NO: 20 - protein, 110 aa, Artificial Sequence
  Synthetic Construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 21 - protein, 112 aa, Artificial Sequence
  Synthetic Construct
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVT

SEQ ID NO: 22 - protein, 110 aa, Artificial Sequence
  Synthetic Construct
EIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEI

SEQ ID NO: 23 - protein, 119 aa, Artificial Sequence
  Synthetic Construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 24 - protein, 105 aa, Artificial Sequence
  Synthetic Construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR

SEQ ID NO: 25 - protein, 5 aa, Artificial Sequence
  Synthetic Construct
GGGGS

SEQ ID NO: 26 - protein, 12 aa, Artificial Sequence
  Synthetic Construct
EPKSCDKTHTCP

SEQ ID NO: 27 - protein, 515 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEPKSCDKTHTCPEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 28 - protein, 503 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 29 - protein, 515 aa, Artificial Sequence
  Synthetic Construct
QIQLVQSGPELKKPGETVKISCKASGYTFTEYPIHWVKQAPGKGFKWMGMIYTDIGKPTYAEEFGRFAFSLETSASTAYLQINNLKNEDTATYFCVRDRYDSLFDYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKEPKSCDKTHTCPEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 30 - protein, 515 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQIQLVQSGPELKKPGETVKISCKASGYTFTEYPIHWVKQAPGKGFKWMGMIYTDIGKPTYAEEFGRFAFSLETSASTAYLQINNLKNEDTATYFCVRDRYDSLFDYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKEPKSCDKTHTCP

SEQ ID NO: 31 - protein, 522 aa, Artificial Sequence
  Synthetic Construct
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLGEPKSCDKTHTCPEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 32 - protein, 522 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPEVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLGEPKSCDKTHTCP

SEQ ID NO: 33 - protein, 501 aa, Artificial Sequence
  Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 34 - protein, 501 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 35 - protein, 502 aa, Artificial Sequence
  Synthetic Construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 36 - protein, 502 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 37 - protein, 518 aa, Artificial Sequence
  Synthetic Construct
  misc_feature (20)..(20): Xaa can be any naturally occurring amino acid
MARFSSSSLDLNWYSLGLQXKLSCKASGYTFTYFDINWLRQRPEQGLEWIGVISPGDGNTNYNENFKGKATLTIDKSSTTAYIQLSRLTSEDSAVYFCARDGNFPYYAMDSWGQGTSVTVSSAKTTPKLEEGEFSEARVDIVMTQIPLSLPVILGDQASISCRSSQSLVYSNGNTYLHWFLQKPGQSPKLLIYNVSNLFSGVPDRFSGSGSGTDFTLKISRVEAEDLGIYFCSQSTHVPTFGGGTKLEIKRADAAAAGSEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 38 - protein, 518 aa, Artificial Sequence
  Synthetic Construct
  misc_feature (279)..(279): Xaa can be any naturally occurring amino acid
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPMARFSSSSLDLNWYSLGLQXKLSCKASGYTFTYFDINWLRQRPEQGLEWIGVISPGDGNTNYNENFKGKATLTIDKSSTTAYIQLSRLTSEDSAVYFCARDGNFPYYAMDSWGQGTSVTVSSAKTTPKLEEGEFSEARVDIVMTQIPLSLPVILGDQASISCRSSQSLVYSNGNTYLHWFLQKPGQSPKLLIYNVSNLFSGVPDRFSGSGSGTDFTLKISRVEAEDLGIYFCSQSTHVPTFGGGTKLEIKRADAAAAGS

SEQ ID NO: 39 - protein, 504 aa, Artificial Sequence
  Synthetic Construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSAGGGGSGGGGSGGGGSSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 40 - protein, 504 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSAGGGGSGGGGSGGGGSSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 41 - protein, 500 aa, Artificial Sequence
  Synthetic Construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 42 - protein, 500 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 43 - protein, 501 aa, Artificial Sequence
  Synthetic Construct
QVQLVQSGGGVVQPGRSLRLSCAASGFTFSSYGLHWVRQAPGKGLEWYAAISYDGSKKYYADSVKGRLTISRDNSKNTLYLQMNSLSPEDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVLEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 44 - protein, 501 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGGGVVQPGRSLRLSCAASGFTFSSYGLHWVRQAPGKGLEWYAAISYDGSKKYYADSVKGRLTISRDNSKNTLYLQMNSLSPEDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 45 - protein, 496 aa, Artificial Sequence
  Synthetic Construct
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVTGGGGSGGGGSGGGGSEIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEIEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 46 - protein, 496 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVTGGGGSGGGGSGGGGSEIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEI

SEQ ID NO: 47 - protein, 498 aa, Artificial Sequence
  Synthetic Construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKREPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 48 - protein, 498 aa, Artificial Sequence
  Synthetic Construct
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR

SEQ ID NO: 49 - protein, 22 aa, Artificial Sequence
  Synthetic Construct
MDFQVQIFSFLLISASVIMSRG

SEQ ID NO: 50 - protein, 525 aa, Artificial Sequence
  Synthetic Construct
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 51 - protein, 525 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 52 - protein, 537 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQIQLVQSGPELKKPGETVKISCKASGYTFTEYPIHWVKQAPGKGFKWMGMIYTDIGKPTYAEEFGRFAFSLETSASTAYLQINNLKNEDTATYFCVRDRYDSLFDYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKEPKSCDKTHTCP

SEQ ID NO: 53 - protein, 525 aa, Artificial Sequence
  Synthetic Construct
MDFQVQIFSFLLISASVIMSRGQIQLVQSGPELKKPGETVKISCKASGYTFTEYPIHWVKQAPGKGFKWMGMIYTDIGKPTYAEEFGRFAFSLETSASTAYLQINNLKNEDTATYFCVRDRYDSLFDYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 54 - protein, 532 aa, Artificial Sequence
  Synthetic Construct
MDFQVQIFSFLLISASVIMSRGEVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLGEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 55 - protein, 537 aa, Artificial Sequence; identical to SEQ ID NO: 52
  Synthetic Construct

SEQ ID NO: 56 - protein, 523 aa, Artificial Sequence
  Synthetic Construct
MDFQVQIFSFLLISASVIMSRGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 57 - protein, 523 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 58 - protein, 524 aa, Artificial Sequence
  Synthetic Construct
MDFQVQIFSFLLISASVIMSRGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 59 - protein, 524 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 60 - protein, 540 aa, Artificial Sequence
  Synthetic Construct
  misc_feature (42)..(42): Xaa can be any naturally occurring amino acid
MDFQVQIFSFLLISASVIMSRGMARFSSSSLDLNWYSLGLQXKLSCKASGYTFTYFDINWLRQRPEQGLEWIGVISPGDGNTNYNENFKGKATLTIDKSSTTAYIQLSRLTSEDSAVYFCARDGNFPYYAMDSWGQGTSVTVSSAKTTPKLEEGEFSEARVDIVMTQIPLSLPVILGDQASISCRSSQSLVYSNGNTYLHWFLQKPGQSPKLLIYNVSNLFSGVPDRFSGSGSGTDFTLKISRVEAEDLGIYFCSQSTHVPTFGGGTKLEIKRADAAAAGSEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 61 - protein, 540 aa, Artificial Sequence
  Synthetic Construct
  misc_feature (301)..(301): Xaa can be any naturally occurring amino acid
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPMARFSSSSLDLNWYSLGLQXKLSCKASGYTFTYFDINWLRQRPEQGLEWIGVISPGDGNTNYNENFKGKATLTIDKSSTTAYIQLSRLTSEDSAVYFCARDGNFPYYAMDSWGQGTSVTVSSAKTTPKLEEGEFSEARVDIVMTQIPLSLPVILGDQASISCRSSQSLVYSNGNTYLHWFLQKPGQSPKLLIYNVSNLFSGVPDRFSGSGSGTDFTLKISRVEAEDLGIYFCSQSTHVPTFGGGTKLEIKRADAAAAGS

SEQ ID NO: 62 - protein, 526 aa, Artificial Sequence
  Synthetic Construct
MDFQVQIFSFLLISASVIMSRGQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSAGGGGSGGGGSGGGGSSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 63 - protein, 526 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSAGGGGSGGGGSGGGGSSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 64 - protein, 522 aa, Artificial Sequence
  Synthetic Construct
MDFQVQIFSFLLISASVIMSRGQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 65 - protein, 522 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 66 - protein, 523 aa, Artificial Sequence
  Synthetic Construct
MDFQVQIFSFLLISASVIMSRGQVQLVQSGGGVVQPGRSLRLSCAASGFTFSSYGLHWVRQAPGKGLEWYAAISYDGSKKYYADSVKGRLTISRDNSKNTLYLQMNSLSPEDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVLEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 67 - protein, 523 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGGGVVQPGRSLRLSCAASGFTFSSYGLHWVRQAPGKGLEWYAAISYDGSKKYYADSVKGRLTISRDNSKNTLYLQMNSLSPEDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 68 - protein, 518 aa, Artificial Sequence
  Synthetic Construct
MDFQVQIFSFLLISASVIMSRGQVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVTGGGGSGGGGSGGGGSEIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEIEPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 69 - protein, 518 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVTGGGGSGGGGSGGGGSEIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEI

SEQ ID NO: 70 - protein, 520 aa, Artificial Sequence
  Synthetic Construct
MDFQVQIFSFLLISASVIMSRGQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKREPKSCDKTHTCPMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTV

SEQ ID NO: 71 - protein, 520 aa, Artificial Sequence
  Synthetic Construct
MDFQVQIFSFLLISASVIMSRGMADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVEPKSCDKTHTCPQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR